(19) United States
(12) Patent Application Publication
(10) Pub. No.: US 2024/0308752 A1
(43) Pub. Date: Sep. 19, 2024

(54) BACTERIAL CELLULOSE FOR FOOD PACKAGING

(71) Applicant: The Chinese University of Hong Kong, Shatin (CN)

(72) Inventors: To NGAI, Tai Wai (CN); Ka Man CHEUNG, Mui Wo (CN); Zhuolun JIANG, Fanling (CN); Sze Man HUNG, Shatin (CN)

(21) Appl. No.: 18/434,775

(22) Filed: Feb. 6, 2024

Related U.S. Application Data
(60) Provisional application No. 63/443,943, filed on Feb. 7, 2023.

(51) Int. Cl. B65D 81/127; C08K 13/02; C12P 19/04

(52) U.S. Cl. CPC B65D 81/1275; C08K 13/02; C12P 19/04

(57) ABSTRACT

Provided herein are biodegradable kombucha bacterial cellulose-based composite materials particularly suitable for formulating films for use in packaging applications, such as food packaging, thereby serving as a replacement for non-degradable plastics. The films are edible, strong, transparent, and have low hygroscopicity. In addition to the bacterial cellulose, the films can also include protein encapsulated within the cellulose matrix, and a crosslinked coating with ionic polysaccharide, a polyether, and a metal cation. Alternatively, the films can include a bacterial cellulose matrix and a hydrophobically modified polysaccharide on the bacterial cellulose matrix. Also provided are an effective, green, and low-cost strategy for fabricating the biodegradable materials.

### CROSS-REFERENCES TO RELATED APPLICATIONS

The present application claims priority to U.S. Provisional Application No. 63/443,943, filed Feb. 7, 2023, the full disclosure of which is incorporated by reference in its entirety for all purposes.

### BACKGROUND

Plastic materials have become an integral part of modern life. However, the widespread use of petroleum-based plastics for food packaging has created a number of significant problems that must be addressed. One major issue with using petroleum-based plastics for food packaging is that these materials are neither biodegradable nor compostable. Plastic waste in landfills can require as long as hundreds of years to break down, and even then, the plastic waste does not fully decompose. Accordingly, every piece of plastic that has ever been produced is still in existence in some form or another. As a result, plastic accumulates in the environment, where it can have serious hazardous consequences for wildlife and ecosystems. Another problem with petroleum-based plastics is that they are made from non-renewable resources. Because oil, the main ingredient in plastic, is a finite resource, reliance on petroleum-based plastics is not sustainable in the long term. In addition, the production of plastic requires a significant amount of energy, contributing to climate change and further degrading the environment. There are also health concerns associated with the use of petroleum-based plastics for food packaging. Some research has suggested that these plastics can leach chemicals into the food that they are storing, where these chemicals can be harmful to humans if ingested. This is especially concerning for foods that are stored in plastic for extended periods of time, as the chemicals may be present in higher concentrations.

Over the past few decades, scientists have investigated biological, bio-derived, and biodegradable polymers as replacements for traditional petroleum-based plastics. From among these bio-based polymers, bacterial cellulose has particular potential as a raw material for use as a film in food packaging applications. This potential is the result of generally favorable physical properties, high purity, non-toxicity, and easy availability of bacterial cellulose. Despite these benefits, there are several drawbacks to using bacterial cellulose for food packaging.

One of the main shortcomings of using bacterial cellulose for food packaging is that this material typically is not as strong as traditional plastics. Because bacterial cellulose may not be able to withstand the same amount of wear and tear as petroleum-based plastics, the bacterial cellulose may be less durable and practical for certain types of food packaging applications. In addition, bacterial cellulose may not be as resistant to moisture and other environmental factors as traditional plastics, which could further affect its performance as a food packaging material. Another drawback of food packaging use of bacterial cellulose is that it is not as transparent as traditional plastics. This could make it difficult to see the food that is being packaged, which could also be a disadvantage or problem for certain types of products and tactics for their marketing. There are also concerns related to the production of bacterial cellulose for food packaging. While bacterial cellulose can be produced in large quantities, it is currently more expensive to produce than traditional petroleum-based plastics. As a result, it may not be a cost-effective alternative for many food packaging applications. The production of bacterial cellulose can also require specific conditions and resources which may not be readily available in all manufacturing locations.

Thus, improved bacterial cellulose modifications and production processes are needed for this material to realize its potential more fully as a promising alternative to traditional petroleum-based plastics. The present disclosure addresses these and other needs by providing compositions and methods related to bacterial cellulose composites having several beneficial advantages for use as food packaging films.

### BRIEF SUMMARY

The present disclosure generally relates to an edible, strong, and transparent bacterial cellulose-based, e.g., kombucha bacterial cellulose-based, composite material that can be effectively and easily fabricated using a green and low-cost biosynthetic strategy. The composite material can incorporate readily available and environmentally friendly components, such as soy protein isolate from a culture medium, and calcium alginate-polyethylene glycol (PEG) or lauryl gallate modified chitosan (CT-LG) as an oil and water resistant coating. The fabricated bio-based composite material advantageously can be completely oil-resistant, 100% safe for humans, and entirely degradable within as little as 1-2 months. The provided composition thus has high potential in food packaging and other value-added fields for the replacement of non-degradable plastics.

In one aspect, the disclosure is to a biodegradable film. The film includes a bacterial cellulose matrix, protein entrapped within the bacterial cellulose matrix, and a physical, e.g., crosslinked, coating on the bacterial cellulose matrix. The (crosslinked) physical coating includes an ionic polysaccharide, a polyether, and a metal cation.

In another aspect, the disclosure is to another biodegradable film. The film includes a bacterial cellulose matrix and a hydrophobic coating on the bacterial cellulose matrix. The hydrophobic coating includes hydrophobically modified polysaccharide.

In another aspect, the disclosure is to a packaging material. The packaging material includes a biodegradable film as described herein.

In another aspect, the disclosure is to a drinking straw. The drinking straw includes a biodegradable film as described herein.

In another aspect, the disclosure is to a method of producing a biodegradable film. The method includes culturing a symbiotic colony of bacteria and yeast (SCOBY) in a cultivation medium. The culturing occurs under culture conditions suitable for synthesis of a bacterial cellulose matrix entrapping protein. The cultivation medium includes a sugar, a tea, and a protein mixture including the protein to be entrapped by the bacterial cellulose matrix. The method further includes harvesting the bacterial cellulose matrix. The method further includes coating the bacterial cellulose matrix with a coating solution. The coating occurs under conditions suitable for forming coated bacterial cellulose. The coating solution includes an ionic polysaccharide and a polyether. The method further includes contacting the coated bacterial cellulose with a crosslinking solution, thereby forming the biodegradable film. The contacting occurs under conditions suitable for crosslinking the coated bacterial cellulose. The crosslinking solution includes a metal salt.

In another aspect, the disclosure is to another method of producing a biodegradable film. The method includes culturing a symbiotic colony of bacteria and yeast (SCOBY) in a cultivation medium. The culturing occurs under culture conditions suitable for synthesis of a bacterial cellulose matrix. The cultivation medium includes a sugar and a tea. The method further includes harvesting the bacterial cellulose matrix. The method further includes coating the bacterial cellulose matrix with a coating solution. The coating occurs under conditions suitable for forming coated bacterial cellulose. The coating solution includes a hydrophobically modified polysaccharide.

### BRIEF DESCRIPTION OF THE DRAWINGS

FIG. 1 is a schematic illustration of an overall composite film preparation process in accordance with a provided embodiment, with soy protein isolate (SPI) as a culture medium additive, and calcium alginate (CA)-polyethylene glycol (PEG) as a physical coating for kombucha bacterial cellulose (KBC).

FIG. 2 is a photograph of a flat and smooth kombucha bacterial cellulose film collected from a petri dish after purification and drying in accordance with a provided embodiment.

FIG. 3 is a photograph of a large-scale kombucha bacterial cellulose film collected after cultivating a culture for 7 days at 30° C. in accordance with a provided embodiment.

FIG. 4 is a photograph of a composite material (KBCS-Alg-PEG200) of kombucha bacterial cellulose produced using a culture medium with SPI (KBCS) coated with alginate-PEG200 and crosslinked with calcium alginate (Alg) in large scale in accordance with a provided embodiment.

FIG. 5 is a schematic illustration of an overall composite preparation process in accordance with a provided embodiment, using chitosan modified by lauryl gallate (CT-LG) as a physical composite coating for KBC, thereby producing a BC/CT-LG material exhibiting excellent hydrophobic properties.

FIG. 6 presents a reaction mechanism for entrapment of lauryl gallate (LG) oligomers in the chitosan slurry.

FIG. 7 presents a reaction mechanism for the coupling of lauryl gallate to chitosan through the formation of irreversible bonds.

FIG. 8 is a graph plotting the number of free bacteria (with Gluconacetobacter kombuchae as dominant species) in cultures having 0, 0.40%, 1.00% and 2.00% added soy protein isolate.

FIG. 9 is a graph plotting the laccase activity of a control group and CT-LG group at pH 4.5 against time.

FIG. 10 is a graph plotting FTIR-ATR spectra of an LG dimer powder, a CT-LG film, and a CT film.

FIG. 11 is a graph plotting an MS spectra of a CT-LG film as measured by MALDI-TOF.

FIG. 12 presents a ¹H-NMR spectrum of a CT-LG film, and a chemical structure identifying hydrogen atoms corresponding to peaks of spectrum.

FIG. 13 presents a ¹H-NMR spectrum of chitosan, and a chemical structure identifying hydrogen atoms corresponding to peaks of spectrum.

FIG. 14 is a graph plotting the oil penetration properties of KBCS and KBC-Alg-PEG200 films.

FIG. 15 is a photograph demonstrating application of the provided bacterial cellulose composite material in a food packaging bag for containing an egg tart.

FIG. 16 is a photograph demonstrating application of the provided bacterial cellulose composite material in a food packaging bag for containing a sweetheart cake.

FIG. 17 is a photograph demonstrating application of the provided bacterial cellulose composite material in a food packaging bag for containing oil.

FIG. 18 is a scanning electron microscope (SEM) image of a cross-section view of a KBCS-Alg-PEG200 coated composite film, showing a smooth surface.

FIG. 19 is an SEM image of a front view of a KBCS-Alg-PEG200 coated composite film, showing no pore structure.

FIG. 20 is an SEM image of a cross-section view of a KBCS uncoated composite film, showing a rough surface.

FIG. 21 is an SEM image of a front view of a KBCS uncoated composite film, showing noticeable pore structure.

FIG. 22 is a photograph of a KBCS film after 15 min of water immersion, with the black bar indicating the enlarged thickness of the film due to swelling.

FIG. 23 is a photograph of a KBCS-Alg-PEG200 film after 15 min of water immersion, with no enlarged thickness of the film due to swelling observed.

FIG. 24 is a graph plotting the water contact angle over time for films of KBC, KBCS, and KBCS with an alginate or alginate-PEG coating. The images within the graph illustrate the water contact angle of KBCS-Alg-PEG200 at Is, 10 min, 15 min, and 25 min.

FIG. 25 is a graph plotting the acid solubility of BC/CT-LG at pH=1-7.

FIG. 26 is a graph plotting the dry state and wet state tensile strengths for films of KBC, KBCS, and KBCS with an alginate or alginate-PEG coating.

FIG. 27 is a graph plotting the dry state and wet state percent elongation at break for films of KBC, KBCS, and KBCS with an alginate or alginate-PEG coating.

FIG. 28 is a graph plotting the dry state and wet state tensile strengths for films of CT, BC, CT-LG, CT/CT-LG, bBC/CT-LG, and BC/CT-LG.

FIG. 29 is a graph plotting the dry state and wet state percent elongation at break for films of CT, BC, CT-LG, CT/CT-LG, bBC/CT-LG, and BC/CT-LG.

FIG. 30 is a graph plotting the dry state and wet state flexural strength for BC and BC/CT-LG drinking straws.

FIG. 31 is a graph plotting a Stress comparison of cereal with no cover, KBCS, KBCS-Alg-PEG200. and LDPE after a week storage under 60% R.H, 25° C.

FIG. 32 is a photograph of cereal packaging formed with KBCS, KBCS-Alg-PEG200, and LDPE.

FIG. 33 is a graph plotting the dry state and wet state flexural strength for KBCS and KBCS-Alg-PEG200 drinking straws.

FIG. 34 is a graph plotting the flexural strength for drinking straws exposed to various beverages.

FIG. 35 is a photograph showing the appearance of drinking straws immersed in various beverages.

FIG. 36 is a photograph showing a side view of drinking straws after 24 h immersion and in a dry state.

FIG. 37 is a photograph showing a front view of drinking straws after 24 h immersion and in a dry state.

FIG. 38 is a photograph showing a front view of commercial paper straws after 24 h immersion, with arrows pointing to loosening and swelling of paper fiber.

FIG. 39 is a photograph showing the appearance of drinking straws formed from the materials disclosed herein in accordance with a provided embodiment.

FIG. 40 is a series of photographs showing the appearance of CT, BC, CT-LG, CT/CT-LG, bBC/CT-LG, and BC/CT-LG materials in the shape of films (top) and hollow rods (bottom).

FIG. 41 is a pair of photographs showing the appearance of a BC/CT-LG drinking straw after is (left) and 30 min (right) immersion in water.

FIG. 42 is a pair of photographs showing the appearance of BC/CT-LG packaging bags carrying ketchup after one day (left) and two days (right).

FIG. 43 is a graph plotting the UV-vis transmission spectra for films of KBC, KBCS, and KBCS with an alginate or alginate-PEG coating.

FIG. 44 is a photograph of an A4 size paper printed with “Bacterial cellulose CUHK” and partially covered with a KBCS film.

FIG. 45 is a photograph of an A4 size paper printed with “Bacterial cellulose CUHK” and partially covered with a KBCS-Alg-PEG200 film.

FIG. 46 is a graph plotting the UV-vis absorption spectra of CT, BC, CT-LG, CT/CT-LG, and BC/CT-LG materials.

FIG. 47 is a graph plotting absorbance values measured in an MTT cytotoxicity test of CT, BC, CT-LG, CT/CT-LG, and BC/CT-LG films.

FIG. 48 is a graph plotting the cell viability values measured in an MTT cytotoxicity test of CT, BC, CT-LG, CT/CT-LG, and BC/CT-LG films.

FIG. 49 is a photograph of results of a degradation test of KBCS and KBCS-Alg-PEG200 films after burial in soil for 1 d.

FIG. 50 is a photograph of results of a degradation test of KBCS and KBCS-Alg-PEG200 films after burial in soil for 10 d.

FIG. 51 is a photograph of results of a degradation test of KBCS and KBCS-Alg-PEG200 films after burial in soil for 20 d.

FIG. 52 is a photograph of results of a degradation test of BC/CT-LG films after burial in soil for 1 d (1), 2 d (2), and 20 d (3).

### DETAILED DESCRIPTION

### I. General

The present disclosure provides a composite bio-derived film based on kombucha bacterial cellulose. The bio-derived material is degradable in the natural environment and can advantageously exhibit desirable properties similar to those of traditional petroleum-based plastics. These beneficial properties include good transparency, high flexibility, high oil resistance, low water hygroscopicity, low weight, and low manufacturing cost. The characteristics of the provided film allow it to be particularly useful as a packaging material, and more particularly as a food packaging material, e.g., a disposable food packaging material. The provided film is also specially suited for other applications benefiting from food-safe oil and water resistant materials, e.g., for use in forming a drinking straw.

The kombucha bacterial cellulose used as a raw material the provided composite film is a residue from kombucha tea fermentation. In some embodiments, the cultivation medium for this fermentation need only contain sugar and tea. This is in significant contrast to other bacterial cellulose fermentation media, such as the commonly used Hestrin and Schramm (HS) medium, which can instead be composed of complex components including peptone, yeast extract, citric acid, sodium diphosphate, and others. The complex nature of these components can disadvantageously increase fermentation costs, and process variability. Another benefit of the simplified kombuch bacterial cellulose medium is that its polyphenolic compound content and low pH can inhibit the growth of the competing microorganisms. Accordingly, such undesired culture competition can be inhibited without addition of fungicide or antibiotics agents, further streamlining the production process and saving cost during industrial scale production of the kombucha bacterial cellulose matrix.

To improve the desired properties of biosynthesized bacterial cellulose films, additive materials or polymers can be incorporated into the growing network of bacterial cellulose fibrils, or be used to modify this network after growth. Most commonly, inorganic nanoparticles, including nano-clay, graphene oxide, and carbon nanoparticles are incorporated in the cellulose by electrospinning to improve the conductivity or mechanical strength. The materials and methods of the present disclosure instead rely on organic natural polymers and proteins, for example added into the cultural medium, to enhance product flexibility and rigidity. These additives can be tightly entrapped in the growing ultra-fine fibers of bacterial cellulose and then form a homogeneously intertwisted composite three-dimensional network. In particular embodiments, the addition of soy protein isolate into the kombucha bacterial cellulose matrix by physical entrapment or through incubation surprisingly promotes enhanced mechanical properties of the network.

Previous reports indicated that an inter penetration between calcium alginate and a bacterial cellulose network could enhance the water resistance and mechanical strength of a bacterial cellulose structure, thus strengthening interlayer hydrogen-bonding and increasing the strength, modulus, and toughness of the complexed material. However, when calcium alginate contacts water, the alginate swells as a hydrogel and causes the wet strength of the composite material to drop considerably. Some compositions disclosed herein surprisingly addresses this disadvantage by including a polyether such as polyethylene glycol. Polyethylene glycol is a widely used non-toxic additive in medicines and cosmetics as a suspension agent and thickener. In some provided film materials, polyethylene glycol can act as a binder for calcium alginate and bacterial cellulose, constructing a compact composite network of these components. The polyethylene glycol or other polyether can also act as a plasticizer in the polymer matrix, decreasing the glass transition temperature (T_{g}), tensile strength, and Young's modulus of the matrix, and increasing the percent elongation at break and toughness of the film.

Another focus of some earlier work involved attempts to improve cellulose structures by grafting long alkyl chains onto the polysaccharide surface. However, most of these previous methods involved toxic reagents and solvents incompatible with or problematic for food safety requirements. An alternative approach relies on a green and non-toxic grafting process for modifying cellulose via laccase catalysis to yield highly hydrophobic surfaces. Laccase is a robust oxidoreductase enzyme with a broad substrate range. The catalyst can function as both a depolymerization and polymerization agent for compounds such as phenols, aminophenol, or polyphenols, resulting in a very attractive combination of compounds that can be attached to a fiber surface. Laccases proved successful for anchoring phenolic substrates to, for example, cellulose, lignin, Kraft pulp, and jute fiber. Furthermore, the grafting of phenol compounds to chitosan surfaces has also been reported. Itzincab-Mejia et al. (Int. J. Food Sci. Technol. 48, (2013): 2034) first introduced enzymatic treatments of chitosan surfaces, providing an attractive alternative to non-specific chemical approaches. Liu et al. (Biomacromolecules 22, (2021): 4501) studied the mechanism and properties of the laccase-catalyzed grafting of lauryl gallate on chitosan surfaces, and conjectured that the resulting material had great potential as a food-packaging material, preservative agent, or edible coating material. Phenolic compounds with different functional groups, including hexyloxyphenol, gallic acid, and lauryl gallate, have been successfully grafted on chitosan surfaces, with some of the resulting composite materials exhibiting enhanced antioxidant and antimicrobial effects. Accordingly, some film materials provided by the present disclosure advantageously incorporate a lauryl gallate modified chitosan coating, which contributes to the beneficial transparency, flexibility, and oil and water resistance of the materials.

### II. Definitions

As used herein, the term “cellulose” refers to a homopolymer of β(1→4) linked D-glucose units that form a linear chain and has the following structure:

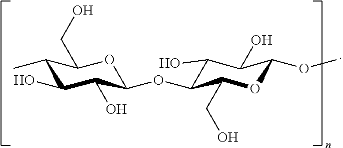

Cellulose can contain several hundred to several thousand or more glucose units, making cellulose a polysaccharide. As used herein, the term “bacterial cellulose” refers to cellulose produced by microorganisms.

As used herein, the term “polysaccharide” refers to a polymeric carbohydrate composed of monosaccharide units bound together by glycosidic linkages. As used herein, the term “ionic polysaccharide refers to any positively or negatively charged polysaccharide, or a salt thereof.

As used here, the term “polyether” refers to a polymer compound composed of units having an ether group separated by aliphatic (that is, by —CH₂—) or aromatic carbon atoms or a combination thereof.

As used herein, the term “plant-derived protein” refers to a protein, protein subunit, or mixture of proteins and/or protein subunits that are each independently encoded by a nucleotide sequence, or coding region, that is expressed within a plant or portion of the plant.

As used herein the term “soy protein isolate” refers to a soybean extract comprising at least about 85%, typically between about 90% and 95%, soy protein on a dry weight basis.

As used herein, the term “alginate” refers to an anionic polysaccharide that is a linear co-polymer with homopolymeric blocks of (1→4) linked mannuronic acid and guluronic acid and has the following structure:

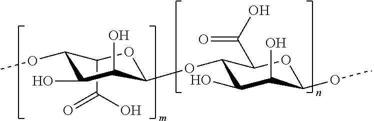

As used herein, the terms “symbiotic colony of bacteria and yeast” and “SCOBY” refer to an often gelatinous material that includes mixture of different strains of bacterial and yeast micorganisms that live together and interact in a mutually beneficial way within the same culture. The bacteria contained in the mixture generally include those that produce lactic acid during fermentation, and/or those that produce acetic acid during fermentation. The yeast contained in the mixture include, but are not limited to any yeasts, including Saccharomyces cerevisiae, Brettanomyces bruxellensis, Candida stellata, Schizosaccharomyces pombe, and Zygosaccharomyces bailii. The bacterial microorganisms of the mixture can belong to the genera Acetobacter, Rhizobium, Agrobacterium, Pseudomonas, Gluconacetobacter, Alcaligenes, Lactobacillus, Lactococcus, Leuconostoc, Bifidobacterium, Thermus, Allobaculum, Ruminococcaceae incertae sedis, Enterococcus, and Propionibacterium. In kombucha, the mixture typically contains a symbiotic culture of Acetobacter bacteria and yeasts such as Saccharomyces and Brettanomyces.

As used herein, the terms “including,” “comprising,” “having,” “containing,” and variations thereof, are inclusive and open-ended and do not exclude additional, unrecited elements or method steps beyond those explicitly recited. As used herein, the phrase “consisting of” is closed and excludes any element, step, or ingredient not explicitly specified. As used herein, the phrase “consisting essentially of” limits the scope of the described feature to the specified materials or steps and those that do not materially affect the basic and novel characteristics of the disclosed feature.

As used herein, the singular forms “a,” “an,” and “the” include plural referents unless the content clearly dictates otherwise. Thus, for example, reference to “a polymer” optionally includes a combination of two or more polymers, and the like.

As used herein, the term “about” denotes a range of value that is +/−10% of a specified value. For instance, “about 10” denotes the value range of 9 to 11 (10+/−1).

### III. Films

In one aspect, the present disclosure provides various biodegradable films that generally include a bacterial cellulose matrix and protein entrapped within the matrix. The films also generally include a coating on the bacterial cellulose matrix, where the coating includes a crosslinked structure of at least one ionic polysaccharide, at least one polyether, and at least one metal cation. In some embodiments, the biodegradable film consists of the bacterial cellulose matrix, the protein, the ionic polysaccharide, the polyether, and the metal cation. In some embodiments, the biodegradable film consists essentially of the bacterial cellulose matrix, the protein, the ionic polysaccharide, the polyether, and the metal cation.

In another aspect, the present disclosure provides a biodegradable film that includes a bacterial cellulose matrix and a hydrophobic coating on the bacterial cellulose matrix. The hydrophobic coating generally includes a hydrophobically modified polysaccharide, e.g., hydrophobically modified chitosan.

The particular combination and relative amounts of these components provide the film with several surprising improvements in various important characteristics, including mechanical strength, oil and water resistance, biodegradability, and transparency. These improved characteristics are particularly advantageous when the provided films are used to replace petroleum-based plastics as packaging materials, and more particularly when used for food packaging.

One advantageously improved property of the provided biodegradable films is its tensile strength while in a wet state. The wet-state tensile strength of a material is important for food packaging because it determines the material's ability to withstand the forces that are applied to it when it is wet, such as when it is exposed to moisture or liquids. A material with high wet-state tensile strength is less likely to break, tear, or deform under these conditions, which can help to preserve the integrity of the food that is being packaged and prevent leaks or spills. This is particularly important for food packaging applications where the packaged food is expected to come into contact with water or other liquids, such as when it is being shipped or stored in a humid environment.

The wet-state tensile strength of the provided biodegradable film can be, for example, between about 50 MPa and about 150 MPa, e.g., between about 50 MPa, and about 97 MPa, between about 56 MPa and about 110 MPa, between about 62 MPa and about 120 MPa, between about 70 MPa and about 130 MPa, or between about 78 MPa and about 150 MPa. In terms of upper limits, the biodegradable film wet-state tensile strength can be, for example, less than about 150 MPa, e.g., less than about 130 MPa, less than about 120 MPa, less than about 110 MPa, less than about 97 MPa, less than about 87 MPa, less than about 78 MPa, less than about 70 MPa, less than about 62 MPa, or less than about 56 MPa. In terms of lower limits, the biodegradable film wet-state tensile strength can be, for example, greater than about 50 MPa, e.g., greater than about 56 MPa, greater than about 62 MPa, greater than about 70 MPa, greater than about 78 MPa, greater than about 87 MPa, greater than about 97 MPa, greater than about 110 MPa, greater than about 120 MPa, or greater than about 130 MPa. Higher wet-state tensile strengths, e.g., greater than about 150 MPa, and lower wet-state tensile strengths, e.g., less than about 50 MPa, are also contemplated. The wet-state tensile strength can be measured according to, for example, the standard procedure of GB/T 1040.3 (2006).

In other embodiments, the wet-state tensile strength of the provided biodegradable film is, for example, between about 20 MPa and about 60 MPa, e.g., between about 29 MPa, and about 44 MPa, between about 24 MPa and about 48 MPa, between about 28 MPa and about 52 MPa, between about 32 MPa and about 56 MPa, or between about 36 MPa and about 60 MPa. In terms of upper limits, the biodegradable film wet-state tensile strength can be, for example, less than about 60 MPa, e.g., less than about 56 MPa, less than about 52 MPa, less than about 48 MPa, less than about 44 MPa, less than about 40 MPa, less than about 36 MPa, less than about 32 MPa, less than about 28 MPa, or less than about 24 MPa. In terms of lower limits, the biodegradable film wet-state tensile strength can be, for example, greater than about 20 MPa, e.g., greater than about 24 MPa, greater than about 28 MPa, greater than about 32 MPa, greater than about 36 MPa, greater than about 40 MPa, greater than about 44 MPa, greater than about 48 MPa, greater than about 52 MPa, or greater than about 56 MPa.

Another advantageously improved property of the provided biodegradable films is its percent elongation at break while in a wet state. Percent elongation at break is a measure of the ductility of a material, which is the ability of the material to deform under tensile stress without rupturing. Materials with high ductility are able to stretch or elongate significantly before breaking. This property is important for food packaging because it allows the material to stretch and conform to the shape of the food without rupturing. This can help to protect the food from damage during handling and transportation. Additionally, materials with high ductility are often more resistant to cracking and breaking, which can also help to extend the shelf life of the packaged food.

The wet-state elongation at break of the provided biodegradable film can be, for example, between about 4% and about 20%, e.g., between about 4% and 13.6%, between about 5.6% and about 15.2%, between about 7.2% and about 16.8%, between about 8.8% and about 18.4%, or between about 10.4% and about 20%. In terms of upper limits, the biodegradable film wet-state elongation at break can be, for example, less than about 20%, e.g., less than about 18.4%, less than about 16.8%, less than about 15.2%, less than about 13.6%, less than about 12%, less than about 10.4%, less than about 8.8%, less than about 7.2%, or less than about 5.6%. In terms of lower limits, the biodegradable film wet-state elongation at break can be, for example, greater than about 4%, e.g., greater than about 5.6%, greater than about 7.2%, greater than about 8.8%, greater than about 10.4%, greater than about 12%, greater than about 13.6%, greater than about 15.2%, greater than about 16.8%, or greater than about 18.4%. Larger wet-state elongations at break, e.g., greater than about 20%, and smaller wet-state elongations at break, e.g., less than about 4%, are also contemplated. The wet-state percent elongation at break can be measured according to, for example, the standard procedure of GB/T 1040.3 (2006).

The provided biodegradable film also beneficially exhibits a low water absorption. Water absorption is a measure of how much water a material can absorb. It is an important property for food packaging because it can affect the shelf life and quality of the food. If a food packaging material has a high water absorption rate, it can cause the food to spoil more quickly by attracting and retaining moisture. This can lead to bacterial growth and other forms of spoilage. Additionally, a material with a high water absorption rate may become weak and brittle when wet, which can make it more prone to breaking or tearing. On the other hand, a material with a low water absorption rate will not absorb as much moisture from the food, which can help to extend its shelf life and maintain its quality. This is especially important for packaged foods that are prone to drying out, such as crackers or chips.

The water absorption of the provided biodegradable film can be, for example, between about 40% and about 200%, e.g., between about 40% and about 110%, between about 47% and about 120%, between about 55% and about 140%, between about 65% and about 170%, or between about 76% and about 200%. In terms of upper limits, the biodegradable film water absorption can be, for example, less than about 200%, e.g., less than about 170%, less than about 140%, less than about 120%, less than about 110%, less than about 89%, less than about 76%, less than about 64%, less than about 55%, or less than about 47%. In terms of lower limits, the biodegradable film water absorption can be, for example, greater than about 40%, e.g., greater than about 47%, greater than about 55%, greater than about 65%, greater than about 76%, greater than about 89%, greater than about 110%, greater than about 120%, greater than about 140%, or greater than about 170%. Higher water absorptions, e.g., greater than 200%, and lower water absorptions, e.g., less than about 40%, are also contemplated. The water absorption can be measured according to, for example, the standard procedure of ASTM D570 (2018).

Another advantage of the provided biodegradable film is a beneficially low water vapor transmission rate. Water vapor transmission rate (WVTR) is a measure of how quickly water vapor can pass through a material. If a food packaging material has a high WVTR, it can allow moisture to pass through the material and come into contact with the food. This can cause the food to spoil more quickly by attracting and retaining moisture. Additionally, a material with a high WVTR may allow the food to dry out more quickly, which can affect its texture and taste. A material instead having a low WVTR will not allow as much moisture to pass through, which can help to extend the shelf life of the packaged food and maintain its quality. This is especially important for foods that are prone to drying out or that are sensitive to changes in humidity. In general, it is important for food packaging materials to have a low WVTR in order to help protect the food from spoilage and maintain its quality.

The water vapor transmission rate of the provided biodegradable film can be, for example, between about 50 g/(m²·hr) and about 200 g/(m²·hr), e.g., between about 50 g/(m²·hr) and about 110 g/(m²·hr), between about 57 g/(m²·hr) and about 130 g/(m²·hr), between about 66 g/(m²·hr) and about 150 g/(m²·hr), between about 76 g/(m²·hr) and about 170 g/(m²·hr), or between about 87 g/(m²·hr) and about 200 g/(m²·hr). In terms of upper limits, the biodegradable film vapor transmission rate can be, for example, less than about 200 g/(m²·hr), e.g., less than about 170 g/(m²·hr), less than about 150 g/(m²·hr), less than about 130 g/(m²·hr), less than about 110 g/(m²·hr), less than about 100 g/(m²·hr), less than about 87 g/(m²·hr), less than about 76 g/(m²·hr), less than about 66 g/(m²·hr), or less than about 57 g/(m²·hr). In terms of lower limits, the biodegradable film water vapor transmission rate can be, for example, greater than about 50 g/(m²·hr), e.g., greater than about 57 g/(m²·hr), greater than about 66 g/(m²·hr), greater than about 76 g/(m²·hr), greater than about 87 g/(m²·hr), greater than about 100 g/(m²·hr), greater than about 110 g/(m²·hr), greater than about 130 g/(m²·hr), greater than about 150 g/(m²·hr), or greater than about 170 g/(m²·hr). Higher water vapor transmission rates, e.g., greater than 200 g/(m²·hr), and lower water vapor transmission rates, e.g., less than 50 g/(m²·hr), are also contemplated. The water vapor transmission rate can be measured according to, for example, the standard procedure of ASTM F1249 (2020).

The provided biodegradable film also advantageously exhibits a water contact angle particularly suitable for food packaging applications. The water contact angle of a material is a measure of its wettability, or the ability of a liquid (such as water) to spread out and contact the surface of a material. This property is important for food packaging because it can affect the way that the food interacts with the packaging material. If a food packaging material has a high water contact angle, it will be more hydrophobic, meaning that it will resist the spread of water and other liquids. This can help to prevent the food from coming into contact with moisture, which can extend shelf life of the food and maintain its quality. Additionally, a material with a high water contact angle may be more resistant to bacterial growth, as bacteria require a moist environment to thrive. A material with a low water contact angle will be more hydrophilic, meaning that it will readily absorb or spread out liquids. This can be beneficial for certain types of food, such as fruits or vegetables, which may benefit from the ability to absorb moisture. However, for other types of food, a low water contact angle may not be desirable, as it can lead to the food coming into contact with moisture and potentially spoiling more quickly.

The water contact angle of the provided biodegradable film can be, for example, between about 65 degrees and about 135 degrees, e.g., between about 65 degrees and about 107 degrees, between about 72 degrees and about 114 degrees, between about 79 degrees and about 121 degrees, between about 86 degrees and about 128 degrees, or between about 93 degrees and about 135 degrees. In terms of upper limits, the biodegradable film water contact angle can be less than about 135 degrees, e.g., less than about 128 degrees, less than about 121 degrees, less than about 114 degrees, less than about 107 degrees, less than about 100 degrees, less than about 93 degrees, less than about 86 degrees, less than about 79 degrees, or less than about 72 degrees. In terms of lower limits, the biodegradable film water contact angle can be, for example, greater than about 65 degrees, e.g., greater than about 72 degrees, greater than about 79 degrees, greater than about 86 degrees, greater than about 93 degrees, greater than about 100 degrees, greater than about 107 degrees, greater than about 114 degrees, greater than about 121 degrees, or greater than about 128 degrees. Larger water contact angles, e.g., greater than about 135 degrees, and smaller water contact angles, e.g., less than about 65 degrees, are also contemplated. The water contact angle can be measured according to, for example, the standard procedure of ASTM D5946 (2017).

Another advantage of the provided biodegradable film is that it a high visible light transmittance, rendering it highly transparent. The light transmittance of a material is a measure of how much light is able to pass through the material. This property is important for food packaging because it can affect the quality and shelf life of the food. If a food packaging material has a high light transmittance, it will allow more light to pass through the material and reach the food. This can be beneficial for certain types of food, such as fruits or vegetables, which may benefit from exposure to light. Additionally, transparent food packaging materials can be desirable for improving the visibility of the packaged food to consumers, who may base purchase decisions on the visual appearance of the food as indicative of the food's freshness, color, regularity, or other features.

The 600-nm light transmittance of the provided biodegradable film can be, for example, between about 15% and about 55%, e.g., between about 15% and about 39%, between about 19% and about 43%, between about 23% and about 47%, between about 27% and about 51%, or between about 31% and about 55%. In terms of upper limits, the biodegradable film 600-nm light transmittance can be, for example, less than about 55%, e.g., less than about 51%, less than about 47%, less than about 43%, less than about 39%, less than about 35%, less than about 31%, less than about 27%, less than about 23%, or less than about 19%. In terms of lower limits, the biodegradable film 600-nm light transmittance can be, for example, greater than about 15%, e.g., greater than about 19%, greater than about 23%, greater than about 27%, greater than about 31%, greater than about 35%, greater than about 39%, greater than about 43%, greater than about 47%, or greater than about 51%. Higher light transmission, e.g., greater than about 55%, and lower light transmission, e.g., less than about 15%, are also contemplated. The 600-nm light transmittance can be measured according to, for example, the standard procedure of ASTM D1003 (2021).

The base component of the provided biodegradable film is a bacterial cellulose matrix, and more preferably, a bacterial cellulose matrix produced during the fermentation of kombucha tea. Kombucha bacterial cellulose has a number of unique properties making it particularly useful in the provided film. Kombucha bacterial cellulose is highly pure and crystalline, with a high tensile strength and a high degree of water retention. It is also biocompatible and biodegradable, making it an environmentally friendly material.

The amount of the bacterial cellulose within the provided biodegradable film can be selected to improve the mechanical strength of the film. The concentration of the bacterial cellulose matrix in the biodegradable film can be, for example, between about 73 wt % and about 88 wt %, e.g., between about 73 wt % and about 82 wt %, between about 74.5 wt % and about 83.5 wt %, between about 76 wt % and about 85 wt %, between about 77.5 wt % and about 86.5 wt %, or between about 79 wt % and about 88 wt %. In terms of upper limits, the bacterial cellulose matrix concentration in the biodegradable film can be, for example, less than about 88 wt %, e.g., less than about 86.5 wt %, less than about 85 wt %, less than about 83.5 wt %, less than about 82 wt %, less than about 80.5 wt %, less than about 79 wt %, less than about 77.5 wt %, less than about 76 wt %, or less than about 74.5 wt %. In terms of lower limits, the bacterial cellulose matrix concentration in the biodegradable film can be, for example, greater than about 73 wt %, e.g., greater than about 74.5 wt %, greater than about 76 wt %, greater than about 77.5 wt %, greater than about 79 wt %, greater than about 80.5 wt %, greater than about 82 wt %, greater than about 83.5 wt %, greater than about 85 wt %, or greater than about 86.5 wt %. Higher bacterial cellulose matrix concentrations, e.g., greater than about 88 wt %, and lower bacterial cellulose matrix concentrations, e.g., less than about 73 wt %, are also contemplated.

The protein entrapped within the bacterial cellulose matrix of the provided biodegradable film has been shown to advantageously enhance the mechanical properties of the film, improving its durability. When protein is incorporated into a cellulose matrix, it can interact with the cellulose in a number of ways. For example, it can alter the crystallinity and mechanical properties of the cellulose, such as its tensile strength and elasticity. The presence of protein may also affect the water-holding capacity and swelling behavior of the cellulose matrix.

In some embodiments, the protein of the provided biodegradable film includes one or more plant-derived proteins. In some embodiments, the protein consists of one one or more plant-derived proteins. Plant-derived protein can be more environmentally sustainable than animal-derived protein, as it requires less land, water, and energy to produce. Additionally, plant-derived proteins are generally hypoallergenic and do not contain the allergens that are often found in animal-derived proteins, such as milk, eggs, and peanuts. In some embodiments, the protein of the biodegradable film includes soy protein isolate. In some embodiments, the protein of the biodegradable film consists of soy protein isolate. The soy protein isolate of the film can originate as a component of the cultivation medium of the fermentation in which the bacterial cellulose matrix is produced. In this way the bacterial cellulose matrix entraps the soy protein isolate as the bacterial cellulose matrix forms. Beneficially, this is a much simpler process for adding protein to a cellulose matrix than alternative approaches in which a cellulose matrix is modified, e.g., through electrospinning, after the cellulose matrix has been formed.

The amount of the protein within the provided biodegradable film can be selected to provide the film with increased durability. The concentration of the protein within the biodegradable film can be, for example, between about 0.27 wt % and about 0.93 wt %, e.g., between about 0.27 wt % and about 0.67 wt %, between about 0.34 wt % and about 0.73 wt %, between about 0.4 wt % and about 0.8 wt %, between about 0.47 wt % and about 0.86 wt %, or between about 0.53 wt % and about 0.93 wt %. In terms of upper limits, the protein concentration in the biodegradable film can be, for example, less than about 0.93 wt %, e.g., less than about 0.86 wt %, less than about 0.8 wt %, less than about 0.73 wt %, less than about 0.67 wt %, less than about 0.6 wt %, less than about 0.53 wt %, less than about 0.47 wt %, less than about 0.4 wt %, or less than about 0.34 wt %. In terms of lower concentrations, the protein concentration in the biodegradable film can be, for example, greater than about 0.27 wt %, e.g., greater than about 0.34 wt %, greater than about 0.4 wt %, greater than about 0.47 wt %, greater than about 0.53 wt %, greater than about 0.6 wt %, greater than about 0.67 wt %, greater than about 0.73 wt %, greater than about 0.8 wt %, or greater than about 0.86 wt %. Higher protein concentrations, e.g., greater than about 0.93 wt %, and lower protein concentrations, e.g., less than about 0.27 wt %, are also contemplated.

The crosslinked coating of some provided biodegradable films has been shown to provide the films with reduced hygroscopicity, increased flexibility, and improved transparency. To give the crosslinked coating environmentally friendly and biodegradable qualities similar to those of the bacterial cellulose matrix, the coating can be based on an ionic polysaccharide. Examples of ionic polysaccharides suitable for use in the provided biodegradable film include hydrophilic colloidal materials and natural gums, i.e., the ammonium and alkali metal salts of alginic acid and mixtures thereof as well as chitosan, which is a common name for the deacetylated form of chitin. Chitin is a natural product comprising poly-(N-acetyl-D-glucosamine). The alginates can be any of the water-soluble alginates including the alkali metal (sodium, potassium, lithium, rubidium and cesium) salts of alginic acid, as well as the ammonium salt, and the soluble alginates of an organic base such as mono-, di-, or tri-ethanolamine, aniline and alike. Other examples of suitable ionic polysaccharides include carboxymethyl cellulose, xanthan gum, gum arabic, gum tragacanth, locust bean gum, tara gum, carboxymethyl starch, cationic starch, gelatin, gellan, pectin, and carrageenan. In some embodiments, the ionic polysaccharide of the biodegradable film includes one or more alginates. In some embodiments, the ionic polysaccharide of the film consists one or more alginates. In some embodiments, the ionic polysaccharide includes sodium alginate. In some embodiments, the ionic polysaccharide consists of sodium alginate.

The viscosity of the ionic polysaccharide of the provided biodegradable film can be selected for good processing and handling characteristics, as well as improved stability with the other components of the crosslinked coating and film. The viscosity of the ionic polysaccharide can be, for example, between about 40 mPa·s and about 1000 mPa·s, e.g., between about 40 mPa·s and about 275 mPa·s, between about 55 mPa·s and about 380 mPa·s, between about 75 mPa·s and about 525 mPa·s, between about 105 mPa·s and about 725 mPa·s, and between about 145 mPa·s and about 1000 mPa·s. In terms of upper limits, the ionic polysaccharide viscosity can be, for example less than about 1000 mPa·s, e.g., less than about 725 mPa·s, less than about 525 mPa·s, less than about 380 mPa·s, less than about 275 mPa·s, less than 200 mPa·s, less than about 145 mPa·s, less than about 105 mPa·s, less than about 75 mPa·s, or less than about 55 mPa·s. In terms of lower limits, the ionic polysaccharide viscosity can be, for example, greater than about 40 mPa·s, e.g., greater than about 55 mPa·s, greater than about 75 mPa·s, greater than about 105 mPa·s, greater than about 145 mPa·s, greater than about 200 mPa·s, greater than about 275 mPa·s, greater than about 380 mPa·s, greater than about 525 mPa·s, or greater than about 724 mPa·s. Higher viscosities, e.g., greater than about 1000 mPa·s, and lower viscosities, e.g., less than about 40 mPa·s, are also contemplated.

The concentration of the ionic polysaccharide within the biodegradable film can be, for example, between about 7 wt % and about 32 wt %, e.g., between about 7 wt % and about 22 wt %, between about 9.5 wt %, and about 24.5 wt %, between about 12 wt % and about 27 wt %, between about 14.5 wt % and about 29.5 wt %, or between about 17 wt % and about 32 wt %. In terms of upper limits, the ionic polysaccharide concentration in the biodegradable film can be, for example, less than about 32 wt %, e.g., less than about 29.5 wt %, less than about 27 wt %, less than about 24.5 wt %, less than about 22 wt %, less than about 19.5 wt %, less than about 17 wt %, less than about 14.5 wt %, less than about 12 wt %, or less than about 9.5 wt %. In terms of lower limits, the ionic polysaccharide concentration in the biodegradable film can be, for example, greater than about 7 wt %, e.g., greater than about 9.5 wt %, greater than about 12 wt %, greater than about 14.5 wt %, greater than about 17 wt %, greater than about 19.5 wt %, greater than about 22 wt %, greater than about 24.5 wt %, greater than about 27 wt %, or greater than about 29.5 wt %. Higher polysaccharide concentrations, e.g., greater than about 32 wt %, and lower polysaccharide concentrations, e.g., less than about 7 wt %, are also contemplated.

The polyether of the crosslinked coating of the provided biodegradable film is shown herein to improve the flexibility, transparency, and oil and water resistance of the coating and film. Some ionic polysaccharides, such as alginate, are generally brittle and inflexible materials, meaning that they are not very deformable and tend to break or shatter when subjected to stress.

This property can limit use of ionic polysaccharides in applications where flexibility is important. Additionally, alginate and other ionic polysaccharides are generally not transparent materials, as they tend to absorb light and not allow it to pass through easily. However, these ionic polysaccharides can be modified to improve their transparency, flexibility and oil and water resistance by adding a polyether to the coating. In some embodiments, the polyether includes polyethylene glycol. In some embodiments, the polyether consists of polyethylene glycol.

The size, e.g., molecular weight, of the polyether in the crosslinked coating of the provided biodegradable film can be selected to allow the polyether to match pore sizes of the ionic polysaccharide when crosslinked, thereby providing the coating and film with the advantageous properties disclosed herein. The polyether can have a number average molecular weight that is, for example, between about 30 and about 1000, e.g., between about 300 and about 720, between about 370 and about 790, between about 440 and about 860, between about 510 and about 930, or between about 580 and about 1000. In terms of upper limits, the number average molecular weight of the polyether can be, for example, less than about 1000, e.g., less than about 930, less than about 860, less than about 790, less than about 720, less than about 650, less than about 580, less than about 510, less than about 440, or less than about 370. In terms of lower limits, the number average molecular weight of the polyether can be, for example, greater than about 300, e.g., greater than about 370, greater than about 440, greater than about 510, greater than about 580, greater than about 650, greater than about 720, greater than about 790, greater than about 860, or greater than about 930. Higher molecular weights, e.g., greater than about 1000, and lower molecular weights, e.g., less than about 300, are also contemplated.

The amount of the polyether in the coating of the provided biodegradable film can also be selected to provide the film with beneficially improved flexibility, transparency, and oil and water resistance. The concentration of the polyether within the biodegradable film can be, for example between about 3.5 wt % and about 12.5 wt %, e.g., between about 3.5 wt % and about 8.9 wt %, between about 4.4 wt % and about 9.8 wt %, between about 5.3 wt % and about 10.7 wt %, between about 6.2 wt % and about 11.6 wt %, or between about 7.1 wt % and about 12.5 wt %. In terms of upper limits, the protein concentration in the biodegradable film can be, for example, less than about 12.5 wt %, e.g., less than about 11.6 wt %, less than about 10.7 wt %, less than about 9.8 wt %, less than about 8.9 wt %, less than about 8 wt %, less than about 7.1 wt %, less than about 6.2 wt %, less than about 5.3 wt %, or less than about 4.4 wt %. In terms of lower limits, the protein concentration in the biodegradable film can be, for example, greater than about 3.5 wt %, e.g., greater than about 4.4 wt %, greater than about 5.3 wt %, greater than about 6.2 wt %, greater than about 7.1 wt %, greater than about 8 wt %, greater than about 8.9 wt %, greater than about 9.8 wt %, greater than about 10.7 wt %, or greater than about 11.6 wt %. Higher protein concentrations, e.g., greater than about 12.5 wt %, and lower protein concentrations, e.g., less than about 12.5 wt %, are also contemplated.

Just as the addition of a polyether to an ionic polysaccharide can improve properties of the polysaccharide such as flexibility, transparence, and water and oil resistance, the crosslinking of the polysaccharide can also improve these properties. Crosslinking can also provide the ionic polysaccharide with increased strength and stiffness. Preferably, the ionic polysaccharide is crosslinked with a metal cation. Examples of metal cations suitable for use in the provided crosslinked coating and film include magnesium, calcium, strontium, barium, titanium, manganese, iron, tungsten, zinc, yttrium, zirconium, molybdenum, niobium, vanadium, cobalt, lead, gallium, indium, germanium palladium, silver, lanthanum, cerium, praseodymium, neodymium, promethium, samarium, europium, gadolinium, terbium, dysprosium, holmium, erbium, thulium, ytterbium, lutetium, hafnium, platinum, gold, aluminum, silicon, tin, or bismuth. In some embodiments, the metal cation includes calcium. In some embodiments, the metal cation consists of calcium.

The amount of the metal cation in the provided crosslinked coating and biodegradable film can be selected to effectively crosslink the ionic polysaccharide of the coating and provide the film with its desired mechanical and other beneficial properties. The concentration of the metal cation within the biodegradable film can be, for example, between about 0.52 wt % and about 2.9 wt %, e.g., between about 0.52 wt % and about 1.9 wt %, between about 0.76 wt % and about 2.2 wt %, between about 1 wt % and about 2.4 wt %, between about 1.2 wt % and about 2.7 wt %, or between about 1.5 wt % and about 2.9 wt %. In terms of upper limits, the metal cation concentration in the biodegradable film can be, for example, less than about 2.9 wt %, e.g., less than about 2.7 wt %, less than about 2.4 wt %, less than about 2.2 wt %, less than about 1.9 wt %, less than about 1.7 wt %, less than about 1.5 wt %. less than about 1.2 wt %, less than about 1 wt %, or less than about 0.76 wt %. In terms of lower limits, the metal cation concentration in the biodegradable film can be, for example, greater than about 0.52 wt %, e.g., greater than about 0.76 wt %, greater than about 1 wt %, greater than about 1.2 wt %, greater than about 1.5 wt %, greater than about 1.7 wt %, greater than about 1.9 wt %, greater than about 2.2 wt %, greater than about 2.4 wt %, or greater than about 2.7 wt %. Higher metal cation concentrations, e.g., greater than about 2.9 wt %, and lower metal cation concentrations, e.g., less than about 0.52 wt %, are also contemplated.

The particularly useful properties of the provided biodegradable film have been demonstrated to be the result not only of the separate concentrations of individual components of the film, but also of the amounts of the components in relation to one another. Notably, the importance of the component ratios in simultaneously enabling different advantageous characteristics had not been previously appreciated. For example, certain relative amounts of the bacterial cellulose matrix with respect to the protein entrapped within the matrix provide the film with its advantageous features. The mass ratio of the bacterial cellulose matrix to the protein within the biodegradable film can be, for example, between about 75:1 and about 325:1, e.g., between about 75:1 and about 225:1, between about 100:1 and about 250:1, between about 125:1 and about 275:1, between about 150:1 and about 300:1, or between about 175:1 and about 325:1. In terms of upper limits, the mass ratio of the bacterial cellulose matrix to the protein in the biodegradable film can be, for example, less than about 325:1, e.g., less than about 300:1, less than about 275:1, less than about 250:1, less than about 225:1, less than about 200:1, less than about 175:1, less than about 150:1, less than about 125:1, or less than about 100:1. In terms of lower limits, the mass ratio of the bacterial cellulose matrix to the protein in the biodegradable film can be, for example, greater than about 75:1, e.g., greater than about 100:1, greater than about 125:1, greater than about 150:1, greater than about 175:1, greater than about 200:1, greater than about 225:1, greater than about 250:1, greater than about 275:1, or greater than about 300:1. Higher mass ratios, e.g., greater than about 325:1, and lower mass ratios, e.g., less than about 75:1, are also contemplated.

Certain relative amounts of the bacterial cellulose matrix in the biodegradable film to the ionic polysaccharide in the film have also been demonstrated as providing the film disclosed herein with its surprisingly useful properties. The mass ratio of the bacterial cellulose matrix to the ionic polysaccharide within the biodegradable film can be, for example, between about 2:1 and about 12:1, e.g., between about 2:1 and about 8:1, between about 3:1 and about 9:1, between about 4:1 and about 10:1, between about 5:1 and about 11:1, or between about 6:1 and about 12:1. In terms of upper limits, the mass ratio of the bacterial cellulose matrix to the ionic polysaccharide in the biodegradable film can be, for example, less than about 12:1, e.g., less than about 11:1, less than about 10:1, less than about 9:1, less than about 8:1, less than about 7:1, less than about 6:1, less than about 5:1, less than about 4:1, or less than about 3:1. In terms of lower limits, the mass ratio of bacterial cellulose matrix to the ionic polysaccharide in the biodegradable film can be, for example, greater than about 2:1, e.g., greater than about 3:1, greater than about 4:1, greater than about 5:1, greater than about 6:1, greater than about 7:1, greater than about 8:1, greater than about 9:1, greater than about 10:1, or greater than about 11:1. Higher mass ratios, e.g., greater than about 12:1, and lower mass ratios, e.g., less than about 2:1, are also contemplated.

Certain relative amounts of the bacterial cellulose matrix in the biodegradable film to the polyether in the film have also been demonstrated as providing the film disclosed herein with its surprisingly useful properties. The mass ratio of the bacterial cellulose matrix to the polyether within the biodegradable film can be, for example, between about 6:1 and about 26:1, e.g., between about 6:1 and about 18:1, between about 8:1 and about 20:1, between about 10:1 and 22:1, between about 12:1 and 24:1, or between about 14:1 and about 26:1. In terms of upper limits, the mass ratio of the bacterial cellulose matrix to the polyether in the biodegradable film can be, for example, less than about 26:1, e.g., less than about 24:1, less than about 22:1, less than about 20:1, less than about 18:1, less than about 16:1, less than about 14:1, less than about 12:1, less than about 10:1, or less than about 8:1. In terms of lower limits, the mass ratio of the bacterial cellulose matrix to the polyether in the biodegradable film can be, for example, greater than about 6:1, e.g., greater than about 8:1, greater than about 10:1, greater than about 12:1, greater than about 14:1, greater than about 16:1, greater than about 18:1, greater than about 20:1, greater than about 22:1, or greater than about 24:1. Higher mass ratios, e.g., greater than about 26:1, and lower mass ratios, e.g., less than about 6:1, are also contemplated.

Certain relative amounts of the bacterial cellulose matrix in the biodegradable film to the metal cation in the film have also been demonstrated as providing the film disclosed herein with its surprisingly useful properties. The mass ratio of the bacterial cellulose matrix to the metal cation within the biodegradable film can be, for example, between about 25:1 and about 175:1, e.g., between about 24:1 and about 115:1, between about 40:1 and about 130:1, between about 55:1 and about 145:1, between about 70:1 and about 160:1, or between about 85:1 and about 175:1. In terms of upper limits, the mass ratio of the bacterial cellulose matrix to the metal cation in the biodegradable film can be less than about 175:1, e.g., less than about 160:1, less than about 145:1, less than about 130:1, less than about 115:1, less than about 100:1, less than about 85:1, less than about 70:1, less than about 55:1, or less than about 40:1. In terms of lower limits, the mass ratio of the bacterial cellulose matrix to the metal cation in the biodegradable film can be, for example, greater than about 25:1, greater than about 40:1, greater than about 55:1, greater than about 70:1, greater than about 85:1, greater than about 100:1, greater than about 115:1, greater than about 130:1, greater than about 145:1, or greater than about 160:1. Higher mass ratios, e.g., greater than about 175:1, and lower mass ratios, e.g., less than about 25:1, are also contemplated.

Certain relative amounts of the ionic polysaccharide in the biodegradable film to the protein in the film have also been demonstrated as providing the film disclosed herein with its surprisingly useful properties. The mass ratio of the ionic polysaccharide to the protein within the biodegradable film can be, for example, between about 7:1 and about 117:1, e.g., between about 7:1 and about 73:1, between about 18:1 and about 84:1, between about 29:1 and about 95:1, between about 40:1 and about 106:1, or between about 51:1 and about 117:1. In terms of upper limits, the mass ratio of the ionic polysaccharide to the protein in the biodegradable film can be, for example, less than about 117:1, e.g., less than about 106:1, less than about 95:1, less than about 84:1, less than about 73:1, less than about 62:1, less than about 51:1, less than about 40:1, less than about 29:1, or less than about 18:1. In terms of lower limits, the mass ratio of the ionic polysaccharide to the protein in the biodegradable film can be, for example, greater than about 7:1, e.g., greater than about 18:1, greater than about 29:1, greater than about 40:1, greater than about 51:1, greater than about 62:1, greater than about 73:1, greater than 84:1, greater than about 95:1, or greater than about 106:1. Higher mass ratios, e.g., greater than about 117:1, and lower mass ratios, e.g., less than about 7:1, are also contemplated.

Certain relative amounts of the polyether in the biodegradable film to the protein in the film have also been demonstrated as providing the film disclosed herein with its surprisingly useful properties. The mass ratio of the polyether to the protein within the biodegradable film can be, for example, between about 3:1 and about 53:1, e.g., between about 3:1 and about 33:1, between about 8:1 and about 38:1, between about 13:1 and about 43:1, or between about 23:1 and about 53:1. In terms of upper limits, the mass ratio of the polyether to the protein in the biodegradable film can be, for example, less than about 53:1, e.g., less than about 48:1, less than about 43:1, less than about 38:1, less than about 33:1, less than about 28:1, less than about 23:1, less than about 18:1, less than about 13:1, or less than about 8:1. In terms of lower limits, the mass ratio of the polyether to the protein in the biodegradable film can be, for example, greater than about 3:1, e.g., greater than about 8:1, greater than about 13:1, greater than about 18:1, greater than about 23:1, greater than about 28:1, greater than 33:1, greater than about 38:1, greater than about 43:1, or greater than about 48:1. Higher mass ratios, e.g., greater than about 53:1, and lower mass ratios, e.g., less than about 3:1, are also contemplated.

Certain relative amounts of the metal cation in the biodegradable film to the protein in the film have also been demonstrated as providing the film disclosed herein with its surprisingly useful properties. The mass ratio of the metal cation to the protein within the biodegradable film can be, for example, between about 0.5:1 and about 10.5:1, e.g., between about 0.5:1 and about 6.5:1, between about 1.5:1 and about 7.5:1, between about 2.5:1 and about 8.5:1, between about 3.5:1 and about 9.5:1, or between about 4.5:1 and about 10.5:1. In terms of upper limits, the mass ratio of the metal cation to the protein in the biodegradable film can be, for example, less than about 10.5:1, e.g., less than about 9.5:1, less than about 8.5:1, less than about 7.5:1, less than about 6.5:1, less than about 5.5:1, less than about 4.5:1, less than about 3.5:1, less than about 2.5:1, or less than about 1.5:1. In terms of lower limits, the mass ratio of the metal cation to the protein in the biodegradable film can be, for example, greater than about 0.5:1, e.g., greater than about 1.5:1, greater than about 2.5:1, greater than about 3.5:1, greater than about 4.5:1, greater than about 5.5:1, greater than about 6.5:1, greater than about 7.5:1, greater than about 8.5:1, or greater than about 9.5:1. Higher mass ratios, e.g., greater than about 10.5:1, and lower mass ratios, e.g., less than about 0.5:1, are also contemplated.

Certain relative amounts of the ionic polysaccharide in the biodegradable film to the polyether in the film have also been demonstrated as providing the film disclosed herein with its surprisingly useful properties. The mass ratio of the ionic polysaccharide to the polyether within the biodegradable film can be, for example, between about 0.5:1 and about 9.5:1, e.g., between about 0.5:1 and about 5.9:1, between about 1.4:1 and about 6.8:1, between about 2.3:1 and about 7.7:1, between about 3.2:1 and about 8.6:1, or between about 4.1:1 and about 9.5:1. In terms of upper limits, the mass ratio of the ionic polysaccharide to the polyether in the biodegradable film can be, for example, less than about 9.5:1, e.g., less than about 8.6:1, less than about 7.7:1, less than about 6.8:1, less than about 5.9:1, less than about 5:1, less than about 4.1:1, less than about 3.2:1, less than about 2.3:1, or less than about 1.4:1. In terms of lower limits, the mass ratio of the ionic polysaccharide to the polyether in the biodegradable film can be, for example, greater than about 0.5:1, e.g., greater than about 1.4:1, greater than about 2.3:1, greater than about 3.2:1, greater than about 4.1:1, greater than about 5:1, greater than about 5.9:1, greater than about 6.8, greater than about 7.7:1, or greater than about 8.6:1. Higher mass ratios, e.g., greater than about 9.5:1, and lower mass ratios, e.g., less than about 0.5:1, are also contemplated.

Certain relative amounts of the ionic polysaccharide in the biodegradable film to the metal cation in the film have also been demonstrated as providing the film disclosed herein with its surprisingly useful properties. The mass ratio of the ionic polysaccharide to the metal cation within the biodegradable film can be, for example, between about 2:1 and about 62:1, e.g., between about 2:1 and about 16:1, between about 3:1 and about 22:1, between about 4:1 and about 31:1, between about 6:1 and about 44:1, or between about 8:1 and about 62:1. In terms of upper limits, the mass ratio of the ionic polysaccharide to the metal cation in the biodegradable film can be, for example, less than about 62:1, e.g., less than about 44:1, less than about 31:1, less than about 22:1, less than about 16:1, less than about 11:1, less than about 8:1, less than about 6:1, less than about 4:1, or less than about 3:1. In terms of lower limits, the mass ratio of the ionic polysaccharide to the metal cation in the biodegradable film can be, for example, greater than about 2:1, e.g., greater than about 3:1, greater than about 4:1, greater than about 6:1, greater than about 8:1, greater than about 11:1, greater than about 16:1, greater than about 22:1, greater than about 31:1, or greater than about 44:1. Higher mass ratios, e.g., greater than about 62:1, and lower mass ratios, e.g., less than about 2:1, are also contemplated.

Certain relative amounts of the polyether in the biodegradable film to the metal cation in the film have also been demonstrated as providing the film disclosed herein with its surprisingly useful properties. The mass ratio of the polyether to the metal cation within the biodegradable film can be, for example, between about 1:1 and about 26:1, e.g., between about 1:1 and about 7.1:1, between about 1.4:1 and about 9.8:1, between about 1.9:1 and about 14:1, between about 2.7:1 and about 19:1, or between about 3.7:1 and about 26:1. In terms of upper limits, the mass ratio of the polyether to the metal cation can be, for example, less than about 26:1, e.g., less than about 19:1, less than about 14:1, less than about 9.8:1, less than about 7.1:1, less than about 5.1:1, less than about 3.7:1, less than about 2.7:1, less than about 1.9:1, or less than about 1.4:1. In terms of lower limits, the mass ratio of the polyether to the metal cation in the biodegradable film can be, for example, greater than about 1:1, e.g., greater than about 1.4:1, greater than about 1.9:1, greater than about 2.7:1, greater than about 3.7:1, greater than about 5.1:1, greater than about 7.1:1, greater than about 9.8:1, greater than about 14:1, or greater than about 19:1. Higher mass ratios, e.g., greater than about 26:1, and lower mass ratios, e.g., less than about 1:1, are also contemplated.

For embodiments in which the provided biodegradable film includes a hydrophobic coating comprising a hydrophobically modified polysaccharide, the hydrophobically modified polysaccharide can be, for example, a hydrophobically modified chitosan. In some examples, the hydrophobically modified polysaccharide is a polysaccharide modified with a phenolic fatty acid ester, e.g., lauryl gallate. In some embodiments, the molar ratio of the phenolic fatty acid ester to the saccharide subunits of the hydrophobically modified polysaccharide is greater than about 0.7:1, e.g., greater than about 0.79:1, greater than about 0.85:1, greater than about 0.89:1, greater than about 0.92:1, greater than about 0.95:1, greater than about 0.96:1, greater than about 0.97:1, greater than about 0.98:1, or greater than about 0.99:1.

The concentration of the hydrophobic coating within the provided biodegradable film can be, for example, between about 10 wt % and about 45 wt %, e.g., between about 10 wt % and about 25 wt %, between about 12 wt % and about 29 wt %, between about 14 wt % and about 33 wt %, between about 16 wt % and about 39 wt %, or between about 18 wt % and about 45 wt %. In terms of upper limits, the concentration of the hydrophobic coating within the biodegradable film can be, for example, less than about 45 wt %, e.g., less than about 39 wt %, less than about 33 wt %, less than about 29 wt %, less than about 25 wt %, less than about 21 wt %, less than about 18 wt %, less than about 16 wt %, less than about 14 wt %, or less than about 12 wt %. In terms of lower limits, the concentration of the hydrophobic coating within the biodegradable film can be, for example, greater than about 10 wt %, e.g., greater than about 12 wt %, greater than about 14 wt %, greater than about 16 wt %, greater than about 18 wt %, greater than about 21 wt %, greater than about 25 wt %, greater than about 29 wt %, greater than about 33 wt %, or greater than about 39 wt %. Higher hydrophobic coating concentrations, e.g., greater than about 45 wt %, and lower hydrophobic coating concentrations, e.g., less than about 10 wt %, are also contemplated.

The provided biodegradable film can have a thickness selected to be suitable for use as a packaging material, e.g., as a food packaging material. The thickness of the biodegradable film can be, for example, between about 10 μm and about 400 μm, e.g., between about 10 μm and about 91 μm, between about 15 μm and about 130 μm, between about 21 μm and about 190 μm, between about 30 μm and about 280 μm, or between about 43 μm and about 400 μm. In terms of upper limits, the biodegradable film thickness can be, for example, less than about 400 μm, e.g., less than about 280 μm, e.g., less than about 190 μm, less than about 130 μm, less than about 91 μm, less than about 63 μm, less than about 44 μm, less than about 30 μm, less than about 21 μm, or less than about 14 μm. In terms of lower limits, the biodegradable film thickness can be, for example, greater than about 10 μm, e.g., greater than about 14 μm, greater than about 21 μm, greater than about 30 μm, greater than about 44 μm, greater than about 63 μm, greater than about 91 μm, greater than about 130 μm, greater than about 190 μm, or greater than about 280 μm. Larger thicknesses, e.g., greater than about 400 μm, and smaller thicknesses, e.g., less than about 10 μm, are also contemplated.

### IV. Packaging Materials and Drinking Straws

Another aspect of the present disclosure relates to packaging materials and drinking straws. The packaging materials and drinking straws include a biodegradable film having any of the compositions disclosed herein. Because the packaging materials and drinking straws include a film having a provided composition, the packaging materials and drinking straws have the desirable characteristics disclosed in relation to the film, including excellent mechanical strength, high oil and water resistance, biodegradability, and/or transparency. The provided packaging materials are particularly well suited for use as food packaging materials. In other words, in some embodiments, the provided packaging material is a food contact substance. The packaging material may, for example, satisfy all requirements of Section 409 of the United States Federal Food, Drug, and Cosmetic Act, defining a food contact substance as a substance intended for use as a component of materials used in manufacturing, packing, packaging, transporting, or holding of food and not having any technical effect in such food.

In some embodiments, the provided food packaging material or drinking straw includes additional substances in addition to a biodegradable film as disclosed herein. For example, the provided food packaging material or drinking straw can include one or more chain terminators, viscosity modifiers, plasticizers, UV stabilizers, catalysts, other polymers, flame retardants, delusterants, antimicrobial agents, antistatic agents, optical brighteners, extenders, processing aids, talc, mica, gypsum, wollastonite and other commonly used additives known to those of skill in the art. Additional suitable additives may be found in Plastics Additives, An A-Z reference, edited by Geoffrey Pritchard (1998). Suitable stabilizers include, but are not limited to, polyethoxylates (such as the polyethoxylated alkyl phenol Triton X-100), polypropoxylates, block copolymeric polyethers, long chain alcohols, polyalcohols, alkylsulfates, alkyl-sulfonates, alkyl-benzenesulfonates, alkylphosphates, alkyl-phosphonates, alkyl-naphthalene sulfonates, carboxylic acids and perfluoronates. Suitable chain extender compounds include, but are not limited to bis-N-acyl bislactam compounds, isophthaloyl bis-caprolactam (IBC), adipoyl bis-caprolactam (ABC), terphthaloyl bis-caprolactam (TBS), and mixtures thereof.

The packaging material or drinking straw can also include anti-block agents. Inorganic solids, usually in the form of diatomaceous earth, represent one class of materials that can be added to the disclosed packaging material. Non-limiting examples include calcium carbonate, silicon dioxide, magnesium silicate, sodium silicate, aluminum silicate, aluminum potassium silicate, and silicon dioxide are examples of suitable antiblock agents.

The disclosed packaging material or drinking straw can also include a nucleating agent to further improve clarity and enhance oxygen barrier performance. Typically, these agents are insoluble, high melting point species that provide a surface for crystallite initiation. By incorporating a nucleating agent, more crystals are initiated, which are smaller in nature. More crystallites or higher % crystallinity correlates to more reinforcement/higher tensile strength and a more tortuous path for oxygen flux (increased barrier); smaller crystallites decreases light scattering which correlates to improved clarity. Non-limiting examples include calcium fluoride, calcium carbonate, talc and Nylon 2,2.

The provided packaging material or drinking straw can also include organic anti-oxidants in the form of hindered phenols such as, but not limited to, Irganox 1010, Irganox 1076 and Irganox 1098; organic phosphites such as, but not limited to, Irgafos 168 and Ultranox 626; aromatic amines; metal salts from Groups IB, IIB, III, and IV of the periodic table; and metal halides of alkali and alkaline earth metals.

### V. Methods for Preparing Films

Another aspect of the present disclosure relates to methods for preparing the provided films. The particular combinations of steps of the methods have been show to impart the films with the advantageous features discussed above, including excellent mechanical strength, high oil and water resistance, biodegradability, and transparency. The methods generally include culturing a symbiotic colony of bacteria and yeast (SCOBY) in a cultivation medium under culture conditions suitable for synthesis of a bacterial cellulose matrix. In some embodiments, the SCOBY includes Acetobacter bacteria. In some embodiments, the SCOBY of the culture is a kombucha SCOBY. In some embodiments, the SCOBY includes Gluconacetobacter kombuchae bacteria. In some embodiments, the majority of the bacteria in the SCOBY is Gluconacetobacter kombuchae. The use of a kombucha SCOBY in the culture can produce a bacterial cellulose matrix giving the provided films their beneficial characteristics.

In some embodiments, the methods further include selecting the identity and/or the amount of the SCOBY cultured in the cultivation medium. The initial concentration of the SCOBY in the cultivation medium can be, for example, between about 15 g/L and about 150 g/L, e.g., between about 15 g/L and about 60 g/L, between about 19 g/L and about 75 g/L, between about 24 g/L and about 95 g/L, between about 30 g/L and about 120 g/L, or between about 38 g/L and about 150 g/L. In terms of upper limits, the cultivation medium initial SCOBY concentration can be, for example, less than about 150 g/L, e.g., less than about 120 g/L, less than about 95 g/L, less than about 75 g/L, less than about 60 g/L, less than about 47 g/L, less than about 38 g/L, less than about 30 g/L, less than about 24 g/L, or less than about 19 g/L. In terms of lower limits, the cultivation medium initial SCOBY concentration can be, for example, greater than about 15 g/L, e.g., greater than about 19 g/L, greater than about 24 g/L, greater than about 30 g/L, greater than about 38 g/L, greater than about 47 g/L, greater than about 60 g/L, greater than about 75 g/L, greater than about 95 g/L, or greater than about 120 g/L. Higher initial SCOBY concentrations, e.g., greater than about 150 g/L, and lower initial SCOBY concentrations, e.g., less than about 15 g/L, are also contemplated.

The cultivation medium of the provided method can beneficially include very few components, where these components can be readily available at low cost. This simplified cultivation medium advantageously reduces the manufacturing cost of the provided film. Another advantage of the simplified cultivation medium is that fewer complex components are needed, reducing this potential source of process variability. In some embodiments, the cultivation medium includes a sugar, a tea, and a protein to be entrapped within the bacterial cellulose matrix. In some embodiments, the cultivation medium consists of a sugar, a tea, and a protein. In some embodiments, the cultivation medium consists essentially of a sugar, a tea, and a protein.

In some embodiments, the provided method further includes selecting the identity and/or the amount of the one or more sugars in the cultivation medium. The cultivation medium sugar can include, for example and without limitation, one or more monosaccharides, e.g., fructose, glucose, and/or xylose; one or more disaccharides, e.g., sucrose, trehalose, and/or lactose; one or more trisaccharides; one or more polysaccharides; or any combination thereof. In some embodiments, the cultivation medium sugar includes sucrose. In some embodiments, the cultivation medium sugar consists of sucrose.

The concentration, e.g., initial concentration, of the sugar in the cultivation medium can be, for example, between about 30 g/L and about 300 g/L, e.g., between about 30 g/L and about 120 g/L, between about 38 g/L and about 150 g/L, between about 48 g/L and about 190 g/L, between about 60 g/L and about 240 g/L, or between about 75 g/L and about 300 g/L. In terms of upper limits, the cultivation medium sugar concentration can be, for example, less than about 300 g/L, e.g., less than about 240 g/L, less than about 190 g/L, less than about 150 g/L, less than about 120 g/L, less than about 95 g/L, less than about 75 g/L, less than about 60 g/L, less than about 48 g/L, or less than about 38 g/L. In terms of lower limits, the cultivation medium sugar concentration can be, for example, greater than about 30 g/L, e.g., greater than about 38 g/L, greater than about 48 g/L, greater than about 60 g/L, greater than about 75 g/L, greater than about 95 g/L, greater than about 120 g/L, greater than about 150 g/L, greater than about 190 g/L, or greater than about 240 g/L. Higher sugar concentrations, e.g., greater than about 300 g/L, and lower sugar concentrations, e.g., less than about 30 g/L, are also contemplated.

In some embodiments, the provided method further includes selecting the identity and/or the amount of the one or more teas in the cultivation medium. The cultivation medium teas can include, for example and without limitation, black tea, green tea, oolong tea, herbal tea, or any combination thereof. In some embodiments, the tea is added to the cultivation medium in the form of a powder. In some embodiments, the tea is added to the cultivation medium in the form of a liquor. In some embodiments, the cultivation medium tea includes black tea powder. In some embodiments, the cultivation medium tea consists of black tea powder.

The concentration, e.g., initial concentration, of powder tea in the cultivation medium can be, for example, between about 3 g/L and about 30 g/L, e.g., between about 3 g/L and about 12 g/L, between about 3.8 g/L and about 15 g/L, between about 4.8 g/L and about 19 g/L, between about 6 g/L and about 24 g/L, or between about 7.5 g/L and about 30 g/L. In terms of upper limits, the cultivation medium tea concentration can be, for example, less than about 30 g/L, e.g., less than about 24 g/L, less than about 19 g/L, less than about 15 g/L, less than about 12 g/L, less than about 9.5 g/L, less than about 7.5 g/L, less than about 6 g/L, less than about 4.8 g/L, or less than about 3.8 g/L. In terms of lower limits, the cultivation medium tea concentration can be, for example, greater than about 3 g/L, e.g., greater than about 3.8 g/L, greater than about 4.8 g/L, greater than about 6 g/L, greater than about 7.5 g/L, greater than about 9.5 g/L, greater than about 12 g/L, greater than about 15 g/L, greater than about 19 g/L, or greater than about 24 g/L. Higher tea concentrations, e.g., greater than about 30 g/L, and lower tea concentrations, e.g., less than about 3 g/L, are also contemplated.

The presence of the protein in the cultivation medium of the provided method allows the bacterial cellulose matrix formed during the SCOBY culturing to entrap the protein as the matrix is being formed. This can significantly simplify the process of adding protein to the bacterial cellulose matrix, as the need for post-culturing steps to incorporate protein in the matrix is reduced or eliminated. This process simplification is another source of the advantageously reduced manufacturing cost of the provided films.

In some embodiments, the provided method further includes selecting the identity and/or the amount of the protein, e.g., protein mixture, in the cultivation medium. The identity of the protein can be as discussed herein in relation to the provided films. For example, the cultivation medium protein mixture can include or consist of one or more plant-derived proteins. The cultivation medium protein mixture can include or consist of soy protein isolate. The concentration, e.g., initial concentration, of the protein mixture in cultivation medium can be, for example, between about 2 g/L and about 20 g/L, e.g., between about 2 g/L and about 8 g/L, between about 2.5 g/L and about 10 g/L, between about 3.2 g/L and about 13 g/L, between about 4 g/L and about 16 g/L, or between about 5 g/L and about 20 g/L. In terms of upper limits, the cultivation medium protein mixture concentration can be, for example, less than about 20 g/L, e.g., less than about 16 g/L, less than about 13 g/L, less than about 10 g/L, less than about 8 g/L, less than about 6.3 g/L, less than about 5 g/L, less than about 4 g/L, less than about 3.2 g/L, or less than about 2.5 g/L. In terms of lower limits, the cultivation medium protein mixture concentration can be, for example, greater than about 2 g/L, e.g., greater than about 2.5 g/L, greater than about 3.2 g/L, greater than about 4 g/L, greater than about 5 g/L, greater than about 6.3 g/L, greater than about 8 g/L, greater than about 10 g/L, greater than about 13 g/L, or greater than about 16 g/L. Higher protein mixture concentrations, e.g., greater than about 20 g/L, and lower protein mixture concentrations, e.g., less than about 2 g/L, are also contemplated.

In some embodiments, the provided method further includes selecting the temperature of the culture conditions. The culture temperature can influence other significant parameters of the culture, including the rate of the fermentation process and the acidity of the culture environment. The culture temperature can be, for example, between about 20° C. and about 40° C., e.g., between about 20° C. and about 32° C., between about 22° C. and about 34° C., between about 24° C. and about 36° C., between about 26° C. and about 38° C., or between about 28° C. and about 40° C. In terms of upper limits, the culture temperature can be, for example, less than about 40° C., e.g., less than about 38° C., less than about 36° C., less than about 34° C., less than about 32° C., less than about 30° C., less than about 28° C., less than about 26° C., less than about 24° C., less than about 26° C., less than about 24° C., or less than about 22° C. In terms of lower limits, the culture temperature can be, for example, greater than about 20° C., e.g., greater than about 22° C., greater than about 24° C., greater than about 26° C., greater than about 28° C., greater than about 30° C., greater than about 32° C., greater than about 34° C., greater than about 36° C., or greater than about 38° C. Higher culture temperatures, e.g., greater than about 40° C., and lower culture temperatures, e.g., less than about 20° C., are also contemplated.

In some embodiments, the provided method further includes selecting the duration of the culturing. The culture duration can influence parameters such as the acidity of the culture environment and the amount of bacterial cellulose produced. The culture duration can be, for example, between about 2 days and about 22 days, e.g., between about 2 days and about 14 days, between about 4 days and about 16 days, between about 6 days and about 18 days, between about 8 days and about 20 days, or between about 10 days and about 22 days. In terms of upper limits, the culture duration can be, for example, less than about 22 days, e.g., less than about 20 days, less than about 18 days, less than about 16 days, less than about 14 days, less than about 12 days, less than about 10 days, less than about 8 days, less than about 6 days, or less than about 4 days. In terms of lower limits, the culture duration can be, for example, greater than about 2 days, e.g., greater than about 4 days, greater than about 6 days, greater than about 8 days, greater than about 10 days, greater than about 12 days, greater than about 14 days, greater than about 16 days, greater than about 18 days, or greater than about 20 days. Longer culture durations, e.g., greater than about 22 days, and shorter culture durations, e.g., less than about 2 days, are also contemplated.

The provided methods further include harvesting the bacterial cellulose matrix from the culture and coating the bacterial cellulose matrix with a coating solution. In some embodiments, the provided method further includes selecting the identity and/or the amount of the components of the coating solution. The identity of the coating solution components can be as discussed herein in relation to the provided films. For example, the coating solution can include or consist of an ionic polysaccharide and a polyether. In some embodiments, the coating solution ionic polysaccharide includes or consists of one or more alginates. In some embodiments, the coating solution ionic polysaccharide includes or consists of sodium alginate. In some embodiments, the viscosity of the ionic polysaccharide in the coating solution is between about 40 mPa·s and about 1000 mPa·s. In some embodiments, the coating solution polyether includes or consists of polyethylene glycol. In some embodiments, the coating solution polyether has a number average molecular weight that is between about 30 and about 1000.

The concentration of the ionic polysaccharide in the coating solution can be, for example, between about 6 g/L and about 60 g/L, e.g., between about 6 g/L and about 24 g/L, between about 7.6 g/L and about 30 g/L, between about 9.5 g/L and about 38 g/L, between about 12 g/L and about 48 g/L, or between about 15 g/L and about 60 g/L. In terms of upper limits, the coating solution ionic polysaccharide concentration can be, for example, less than about 60 g/L, e.g., less than about 48 g/L, less than about 38 g/L, less than about 30 g/L, less than about 24 g/L, less than about 19 g/L, less than about 15 g/L, less than about 12 g/L, less than about 9.5 g/L, or less than about 7.6 g/L. In terms of lower limits, the coating solution ionic polysaccharide concentration can be, for example, greater than about 6 g/L, e.g., greater than about 7.6 g/L, greater than about 9.5 g/L, greater than about 12 g/L, greater than about 15 g/L, greater than about 19 g/L, greater than about 24 g/L, greater than about 30 g/L, greater than about 38 g/L, or greater than about 48 g/L. Higher ionic polysaccharide concentrations, e.g., greater than about 60 g/L, and lower ionic polysaccharide concentrations, e.g., less than about 6 g/L, are also contemplated.

The concentration of the polyether in the coating solution can be, for example, between about 6 g/L and about 60 g/L, e.g., between about 6 g/L and about 24 g/L, between about 7.6 g/L and about 30 g/L, between about 9.5 g/L and about 38 g/L, between about 12 g/L and about 48 g/L, or between about 15 g/L and about 60 g/L. In terms of upper limits, the coating solution polyether concentration can be, for example, less than about 60 g/L, e.g., less than about 48 g/L, less than about 38 g/L, less than about 30 g/L, less than about 24 g/L, less than about 19 g/L, less than about 15 g/L, less than about 12 g/L, less than about 9.5 g/L, or less than about 7.6 g/L. In terms of lower limits, the coating solution polyether concentration can be, for example, greater than about 6 g/L, e.g., greater than about 7.6 g/L, greater than about 9.5 g/L, greater than about 12 g/L, greater than about 15 g/L, greater than about 19 g/L, greater than about 24 g/L, greater than about 30 g/L, greater than about 38 g/L, or greater than about 48 g/L. Higher polyether concentrations, e.g., greater than about 60 g/L, and lower polyether concentrations, e.g., less than about 6 g/L, are also contemplated.

The mass ratio of the ionic polysaccharide to the polyether in the coating solution can be, for example, between about 1:10 to about 10:1, e.g., between about 1:10 and about 1.6:10, between about 1:6.3 to about 2.5:1, between about 1:4 and about 4:1, between about 1:2.5 and about 6.3:1, or between about 1:1.6 and about 10:1. In terms of upper limits, the mass ratio of the ionic polysaccharide to the polyether in the coating solution can be, for example, less than about 10:1, e.g., less than about 6.3:1, less than about 4:1, less than about 2.5:1, less than about 1.6:1, less than about 1:1, less than about 1:1.6, less than about 1:2.5, less than about 1:4, or less than about 1:6.3. In terms of lower limits, the mass ratio of the ionic polysaccharide to the polyether in the coating solution can be, for example, greater than about 1:10, e.g., greater than about 1:6.3, greater than about 1:4, greater than about 1:2.5, greater than about 1:1.6, greater than about 1:1, greater than about 1.6:1, greater than about 2.5:1, greater than about 4:1, or greater than about 6.3:1. Higher mass ratios, e.g., greater than about 10:1, and lower mass ratios, e.g., less than about 1:10, are also contemplated.

The provided method can further include contacting the coated bacterial cellulose matrix with a crosslinking solution, where the crosslinking solution provides a metal cation suitable for crosslinking the coating on the bacterial cellulose matrix, thereby producing a biodegradable film. The metal cation can be any of those discussed herein in relation to the provided films. The crosslinking solution source of the metal cation can be, for example and without limitation, a metal salt, a transition metal complex, a metal hydroxide, a metal oxide, or any combination thereof. In some embodiments, the provided method further includes selecting the identity and/or the amount of the metal cation source in the crosslinking solution. In some embodiments, the crosslinking solution includes or consists of a metal salt. In some embodiments, the calcium salt includes or consists of calcium lactate.

The concentration of the metal salt in the crosslinking solution can be, for example, between about 6 g/L and about 300 g/L, e.g., between about 6 g/L and about 63 g/L, between about 8.9 g/L and about 93 g/L, between about 13 g/L and about 140 g/L, between about 19 g/L and about 200 g/L, or between about 29 g/L and about 300 g/L. In terms of upper limits, the crosslinking solution metal salt concentration can be, for example, less than about 300 g/L, e.g., less than about 200 g/L, less than about 140 g/L, less than about 93 g/L, less than about 63 g/L, less than about 42 g/L, less than about 29 g/L, less than about 19 g/L, less than about 13 g/L, or less than about 8.9 g/L. In terms of lower limits, the crosslinking solution metal salt concentration can be, for example, greater than about 6 g/L, e.g., greater than about 8.9 g/L, greater than about 13 g/L, greater than about 19 g/L, greater than about 29 g/L, greater than about 42 g/L, greater than about 63 g/L, greater than about 93 g/L, greater than about 140 g/L, or greater than about 200 g/L. Higher metal salt concentrations, e.g., greater than about 300 g/L, and lower metal salt concentrations, e.g., less than about 6 g/L, are also contemplated.

In some embodiments, the provided method further includes preparing a coating solution, wherein the coating solution includes a hydrophobically modified polysaccharide, e.g., a hydrophobically modified chitosan. In some examples, the hydrophobically modified polysaccharide includes a polysaccharide modified with a phenolic fatty acid ester. In particular examples, the provided method further includes forming a reaction mixture containing the polysaccharide, the phenolic fatty acid ester, and an oxidoreductase enzyme, e.g., laccase. The reaction mixture can be maintained under reaction conditions sufficient for the reaction mixture to form the hydrophobically modified polysaccharide.

The reaction conditions can be selected to include a reaction temperature producing a hydrophobically modified polysaccharide with a high degree of substitution. The reaction conditions can include a reaction temperature that is, for example, between about 30° C. and about 50° C., e.g., between about 30° C. and about 42° C., between about 32° C. and about 44° C., between about 34° C. and about 46° C., between about 36° C. and about 48° C., or between about 38° C. and about 50° C. In terms of upper limits, the reaction temperature can be, for example, less than about 50° C., e.g., less than about 48° C., less than about 46° C., less than about 44° C., less than about 42° C., less than about 40° C., less than about 38° C., less than about 36° C., less than about 34° C., or less than about 32° C. In terms of lower limits, the reaction temperature can be, for example, greater than about 30° C., e.g., greater than about 32° C., greater than about 34° C., greater than about 36° C., greater than about 38° C., greater than about 40° C., greater than about 42° C., greater than about 44° C., greater than about 46° C., or greater than about 48° C. Higher reaction temperatures, e.g., greater than about 50° C., and lower reaction temperatures, e.g., less than about 30° C., are also contemplated.

The reaction conditions can be selected to include a reaction duration producing a hydrophobically modified polysaccharide with a high degree of substitution. The reaction conditions can include a reaction duration that is, for example, between about 10 h and about 40 h, e.g., between about 10 h and about 28 h, between about 13 h and about 31 h, between about 16 h and about 34 h, between about 19 h and about 37 h, or between about 22 h and about 40 h. In terms of upper limits, the reaction temperature can be, for example, less than about 40 h, e.g., less than about 37 h, less than about 34 h, less than about 31 h, less than about 28 h, less than about 25 h, less than about 22 h, less than about 19 h, less than about 16 h, or less than about 13 h. In terms of lower limits, the reaction temperature can be, for example, greater than about 10 h, e.g., greater than about 13 h, greater than about 16 h, greater than about 19 h, greater than about 22 h, greater than about 25 h, greater than about 28 h, greater than about 31 h, greater than about 34 h, or greater than about 37 h. Higher reaction temperatures, e.g., greater than about 40 h, and lower reaction temperatures, e.g., less than about 10 h, are also contemplated.

In some embodiments, the provided method further includes drying the biodegradable film. In some embodiments, the method further includes selecting the temperature of the drying of the biodegradable film. The drying temperature can influence properties of the film including strength, water content, and shrinkage. The drying temperature can be, for example, between about 60° C. and about 160° C., e.g., between about 60° C. and about 120° C., between about 70° C. and about 130° C., between about 80° C. and 140° C., between about 90° C. and 150° C., or between about 100° C. and about 160° C. In terms of upper limits, the drying temperature can be less than about 160° C., e.g., less than about 150° C., less than about 140° C., less than about 130° C., less than about 120° C., less than about 110° C., less than about 100° C., less than about 90° C., less than about 80° C., or less than about 70° C. In terms of lower limits, the drying temperature can be greater than about 60° C., e.g., greater than about 70° C., greater than about 80° C., greater than about 90° C., greater than about 100° C., greater than about 110° C., greater than about 120° C., greater than about 130° C., greater than about 140° C., or greater than about 150° C. Higher drying temperatures, e.g., greater than about 160° C., and lower drying temperatures, e.g., less than about 60° C., are also contemplated.

In other embodiments, the drying temperature is between about 40° C. and about 80° C., e.g., between about 40° C. and about 64° C., between about 44° C. and about 68° C., between about 48° C. and about 72° C., between about 52° C. and 76° C., or between about 56° C. and 80° C. In terms of upper limits, the drying temperature can be less than about 80° C., e.g., less than about 76° C., less than about 72° C., less than about 68° C., less than about 64° C., less than about 60° C., less than about 56° C., less than about 52° C., less than about 48° C., or less than about 44° C. In terms of lower limits, the drying temperature can be greater than about 40° C., e.g., greater than about 44° C., greater than about 48° C., greater than about 52° C., greater than about 56° C., greater than about 60° C., greater than about 64° C., greater than about 68° C., greater than about 72° C., or greater than about 76° C.

### VI. Exemplary Embodiments

The following embodiments are contemplated. All combinations of features and embodiments are contemplated.

Embodiment 1: A biodegradable film comprising: a bacterial cellulose matrix; protein entrapped within the bacterial cellulose matrix; and a crosslinked coating on the bacterial cellulose matrix, wherein the crosslinked coating comprises an ionic polysaccharide, a polyether, and a metal cation.

Embodiment 2: An embodiment of embodiment 1, wherein the protein comprises a plant-derived protein.

Embodiment 3: An embodiment of embodiment 2, wherein the plant-derived protein comprises soy protein isolate.

Embodiment 4: An embodiment of any one of embodiments 1-3, wherein the ionic polysaccharide comprises an alginate.

Embodiment 5: An embodiment of embodiment 4, wherein the alginate comprises sodium alginate.

Embodiment 6: An embodiment of any one of embodiments 1-5, wherein the polyether comprises polyethylene glycol.

Embodiment 7: An embodiment of any one of embodiments 1-6, wherein the polyether has a number average molecular weight less than about 1000.

Embodiment 8: An embodiment of any one of embodiments 1-7, wherein the metal cation comprises calcium.

Embodiment 9: An embodiment of any one of embodiments 1-8, wherein the concentration of the bacterial cellulose matrix within the biodegradable film is between about 76 wt % and about 85 wt %.

Embodiment 10: An embodiment of any one of embodiments 1-9, wherein the concentration of the protein within the biodegradable film is between about 0.4 wt % and about 0.8 wt %.

Embodiment 11: An embodiment of any one of embodiments 1-10, wherein the concentration of the ionic polysaccharide within the biodegradable film is between about 12 wt % and about 27 wt %.

Embodiment 12: An embodiment of any one of embodiments 1-11, wherein the concentration of the polyether within the biodegradable film is between about 5.3 wt % and about 10.7 wt %.

Embodiment 13: An embodiment of any one of embodiments 1-12, wherein the concentration of the metal cation within the biodegradable film is between about 1.0 wt % and about 2.4 wt %.

Embodiment 14: An embodiment of any one of embodiments 1-13, wherein the mass ratio of the bacterial cellulose matrix to the protein within the biodegradable film is between about 75:1 and about 225:1.

Embodiment 15: An embodiment of any one of embodiments 1-14, wherein the mass ratio of the bacterial cellulose matrix to the ionic polysaccharide within the biodegradable film is between about 2:1 and about 12:1.

Embodiment 16: An embodiment of any one of embodiments 1-15, wherein the mass ratio of the bacterial cellulose matrix to the polyether within the biodegradable film is between about 6:1 and about 26:1.

Embodiment 17: An embodiment of any one of embodiments 1-16, wherein the mass ratio of the bacterial cellulose matrix to the metal cation within the biodegradable film is between about 25:1 and about 115:1.

Embodiment 18: An embodiment of any one of embodiments 1-17, wherein the mass ratio of the protein to the ionic polysaccharide within the biodegradable film is between about 7:1 and about 73:1.

Embodiment 19: An embodiment of any one of embodiments 1-18, wherein the mass ratio of the protein to the polyether within the biodegradable film is between about 3:1 and about 53:1.

Embodiment 20: An embodiment of any one of embodiments 1-19, wherein the mass ratio of the protein to the metal cation within the biodegradable film is between about 0.5:1 and about 10.5:1.

Embodiment 21: An embodiment of any one of embodiments 1-20, wherein the mass ratio of the ionic polysaccharide to the polyether within the biodegradable film is between about 0.5:1 and about 5.9:1.

Embodiment 22: An embodiment of any one of embodiments 1-21, wherein the mass ratio of the ionic polysaccharide to the metal cation within the biodegradable film is between about 4:1 and about 31:1.

Embodiment 23: An embodiment of any one of embodiments 1-22, wherein the mass ratio of the polyether to the metal cation within the biodegradable film is between about 1.9:1 and about 14:1.

Embodiment 24: An embodiment of any one of embodiments 1-23, wherein the biodegradable film has a thickness between about 10 μm and about 400 μm.

Embodiment 25: An embodiment of any one of embodiments 1-24, wherein the biodegradable film has a wet-state tensile strength greater than about 50 MPa, as measured by GB/T 1040.3 (2006).

Embodiment 26: An embodiment of any one of embodiments 1-25, wherein the biodegradable film has a wet-state elongation at break greater than about 4%, as measured by GB/T 1040.3 (2006).

Embodiment 27: An embodiment of any one of embodiments 1-26, wherein the biodegradable film exhibits complete oil resistance, as measured by TAPPI T507 (2022).

Embodiment 28: An embodiment of any one of embodiments 1-27, wherein the biodegradable film has a water absorption less than about 200%, as measured by ASTM D570 (2018).

Embodiment 29: An embodiment of any one of embodiments 1-28, wherein the biodegradable film has a water vapor transmission rate less than about 200 g/(m²·hr), as measured by ASTM F1249 (2020).

Embodiment 30: An embodiment of any one of embodiments 1-29, wherein the biodegradable film has a water contact angle greater than about 65 degrees, as measured by ASTM D5946 (2017).

Embodiment 31: An embodiment of any one of embodiments 1-30, wherein the biodegradable film has a 600-nm light transmittance greater than about 15%, as measured by ASTM D1003 (2021).

Embodiment 32: A biodegradable film comprising: a bacterial cellulose matrix; and a hydrophobic coating on the bacterial cellulose matrix, wherein the hydrophobic coating comprises a hydrophobically modified polysaccharide.

Embodiment 33: An embodiment of embodiment 32, wherein the biodegradable film further comprises protein entrapped within the bacterial cellulose matrix.

Embodiment 34: An embodiment of embodiment 33, wherein the protein comprises a plant-derived protein.

Embodiment 35: An embodiment of embodiment 43, wherein the plant-derived protein comprises soy protein isolate.

Embodiment 36: An embodiment of any one of embodiments 32-35, wherein the concentration of the protein within the biodegradable film is between about 0.4 wt % and about 0.8 wt %.

Embodiment 37: An embodiment of any one of embodiments 32-36, wherein the hydrophobically modified polysaccharide comprises a hydrophobically modified chitosan.

Embodiment 38: An embodiment of any one of embodiments 32-37, wherein the hydrophobically modified polysaccharide comprises a polysaccharide modified with a phenolic fatty acid ester.

Embodiment 39: An embodiment of embodiment 38, wherein the phenolic fatty acid ester comprises lauryl gallate.

Embodiment 40: An embodiment of embodiment 38 or 39, wherein the molar ratio of the phenolic fatty acid ester to the saccharide subunits of the hydrophobically modified polysaccharide is greater than about 0.7:1.

Embodiment 41: An embodiment of any one of embodiments 32-40, wherein the concentration of the hydrophobic coating within the biodegradable film is between about 10 wt % and about 45 wt %.

Embodiment 42: An embodiment of any one of embodiments 32-41, wherein the biodegradable film has a wet-state tensile strength greater than about 20 MPa, as measured by GB/T 1040.3 (2006).

Embodiment 43: An embodiment of any one of embodiments 32-42, wherein the biodegradable film has a dry-state tensile strength greater than about 50 MPa, as measured by GB/T 1040.3 (2006).

Embodiment 44: An embodiment of any one of embodiments 32-43, wherein the biodegradable film has a water vapor transmission rate less than about 200 g/(m²·hr), as measured by ASTM F1249 (2020).

Embodiment 45: An embodiment of any one of embodiments 32-44, wherein the biodegradable film has a water contact angle greater than about 65 degrees, as measured by ASTM D5946 (2017).

Embodiment 46: A packaging material comprising the biodegradable film of any one of embodiments 1-45.

Embodiment 47: An embodiment of embodiment 46, wherein the packaging material is a food contact substance.

Embodiment 48: A drinking straw comprising the biodegradable film of any one of embodiments 1-45.

Embodiment 49: A method of producing a biodegradable film, the method comprising: culturing a symbiotic colony of bacteria and yeast (SCOBY) in a cultivation medium under culture conditions suitable for synthesis of a bacterial cellulose matrix entrapping protein, wherein the cultivation medium comprises a sugar, a tea, and a protein mixture including the protein; harvesting the bacterial cellulose matrix; coating the bacterial cellulose matrix with a coating solution under conditions suitable for forming coated bacterial cellulose, wherein the coating solution comprises an ionic polysaccharide and a polyether; and contacting the coated bacterial cellulose with a crosslinking solution under conditions suitable for crosslinking the coated bacterial cellulose, wherein the crosslinking solution comprises a metal salt, thereby forming the biodegradable film.

Embodiment 50: An embodiment of embodiment 49, wherein the cultivation medium consists essentially of the sugar, the tea, and the protein mixture.

Embodiment 51: An embodiment of embodiment 49 or 50, wherein the protein mixture comprises a plant-derived protein mixture.

Embodiment 52: An embodiment of any one of embodiments 49-51, wherein the plant-derived protein mixture comprises soy protein isolate.

Embodiment 63: An embodiment of any one of embodiments 49-52, wherein the sugar comprises sucrose.

Embodiment 54: An embodiment of any one of embodiments 49-53, wherein the tea comprises black tea powder.

Embodiment 55: An embodiment of any one of embodiments 49-54, wherein the concentration of the sugar in the cultivation medium is between about 30 g/L and about 300 g/L.

Embodiment 56: An embodiment of any one of embodiments 49-55, wherein the concentration of the protein mixture in the cultivation medium is between about 2 g/L and about 20 g/L.

Embodiment 57: An embodiment of any one of embodiments 49-56, wherein the concentration of the tea in the cultivation medium is between about 3 g/L and about 30 g/L.

Embodiment 58: An embodiment of any one of embodiments 49-57, wherein the initial concentration of the SCOBY in the cultivation medium is between about 15 g/L and about 150 g/L.

Embodiment 59: An embodiment of any one of embodiments 49-58, wherein the culture conditions comprise a culture temperature between about 20° C. and about 40° C.

Embodiment 60: An embodiment of any one of embodiments 49-59, wherein the culture conditions comprise a culture duration between about 2 days and about 22 days.

Embodiment 61: An embodiment of any one of embodiments 49-60, wherein the ionic polysaccharide comprises an alginate.

Embodiment 62: An embodiment of embodiment 61, wherein the alginate comprises sodium alginate.

Embodiment 63: An embodiment of any one of embodiments 49-62, wherein the polyether comprises polyethylene glycol.

Embodiment 67: An embodiment of any one of embodiments 49-63, wherein the polyether has a number average molecular weight less than about 1000.

Embodiment 68: An embodiment of any one of embodiments 49-64, wherein the concentration of the ionic polysaccharide in the coating solution is between about 6 g/L and about 60 g/L.

Embodiment 69: An embodiment of any one of embodiments 49-65, wherein the concentration of the polyether in the coating solution is between about 6 g/L and about 60 g/L.

Embodiment 70: An embodiment of any one of embodiments 49-66, wherein the metal salt comprises a calcium salt.

Embodiment 71: An embodiment of any one of embodiments 57, wherein the calcium salt comprises calcium lactate.

Embodiment 72: An embodiment of any one of embodiments 49-58, wherein the concentration of the metal salt in the crosslinking solution is between about 6 g/L and about 300 g/L.

Embodiment 73: An embodiment of any one of embodiments 49-69, wherein the method further comprises: drying the biodegradable film at a temperature between about 60° C. and about 160° C.

Embodiment 74: A method of producing a biodegradable film, the method comprising: culturing a symbiotic colony of bacteria and yeast (SCOBY) in a cultivation medium under culture conditions suitable for synthesis of a bacterial cellulose matrix, wherein the cultivation medium comprises a sugar and a tea; harvesting the bacterial cellulose matrix; and coating the bacterial cellulose matrix with a coating solution under conditions suitable for forming coated bacterial cellulose, wherein the coating solution comprises a hydrophobically modified polysaccharide.

Embodiment 75: An embodiment of embodiment 74, wherein the cultivation medium further comprises a protein mixture including a protein, and wherein the bacterial cellulose matrix entraps the protein during the culturing of the SCOBY.

Embodiment 76: An embodiment of embodiment 75, wherein the cultivation medium consists essentially of the sugar, the tea, and the protein mixture.

Embodiment 77: An embodiment of embodiment 75 or 76, wherein the protein mixture comprises a plant-derived protein mixture.

Embodiment 78: An embodiment of any one of embodiments 75-77, wherein the plant-derived protein mixture comprises soy protein isolate.

Embodiment 79: An embodiment of any one of embodiments 75-78, wherein the concentration of the protein mixture in the cultivation medium is between about 2 g/L and about 20 g/L.

Embodiment 80: An embodiment of any one of embodiments 74-79, wherein the sugar comprises sucrose.

Embodiment 81: An embodiment of any one of embodiments 74-80, wherein the tea comprises black tea powder.

Embodiment 82: An embodiment of any one of embodiments 74-81, wherein the concentration of the sugar in the cultivation medium is between about 30 g/L and about 300 g/L.

Embodiment 83: An embodiment of any one of embodiments 74-82, wherein the concentration of the tea in the cultivation medium is between about 3 g/L and about 30 g/L.

Embodiment 84: An embodiment of any one of embodiments 74-83, wherein the initial concentration of the SCOBY in the cultivation medium is between about 15 g/L and about 150 g/L.

Embodiment 85: An embodiment of any one of embodiments 74-84, wherein the culture conditions comprise a culture temperature between about 20° C. and about 40° C.

Embodiment 86: An embodiment of any one of embodiments 74-85, wherein the culture conditions comprise a culture duration between about 2 days and about 22 days.

Embodiment 87: An embodiment of any one of embodiments 74-86, wherein the hydrophobically modified polysaccharide comprises a hydrophobically modified chitosan.

Embodiment 88: An embodiment of any one of embodiments 74-87, wherein the hydrophobically modified polysaccharide comprises a polysaccharide modified with a phenolic fatty acid ester.

Embodiment 89: An embodiment of embodiment 88, wherein the phenolic fatty acid ester comprises lauryl gallate.

Embodiment 90: An embodiment of embodiment 88 or 89, wherein the method further comprises preparing the coating solution by forming a reaction mixture comprising the polysaccharide, the phenolic fatty acid ester, and an oxidase enzyme under reaction conditions sufficient for the reaction mixture to form the hydrophobically modified polysaccharide.

Embodiment 91: An embodiment of embodiment 91, wherein the oxidase enzyme comprises laccase.

Embodiment 92: An embodiment of embodiment 90 or 91, wherein the reaction conditions comprise a reaction temperature between about 30° C. and about 50° C.

Embodiment 93: An embodiment of any one of embodiments 90-92, wherein the reaction conditions comprise a reaction duration less than about 40 hours.

Embodiment 94: An embodiment of any one of embodiments 74-93, wherein the method further comprises: drying the biodegradable film at a temperature between about 40° C. and about 80° C.

### EXAMPLES

The present disclosure will be better understood in view of the following non-limiting examples. The following examples are intended for illustrative purposes only and do not limit in any way the scope of the present invention.

### Example 1. Production of a Kombucha Bacterial Cellulose-Based Composite Material

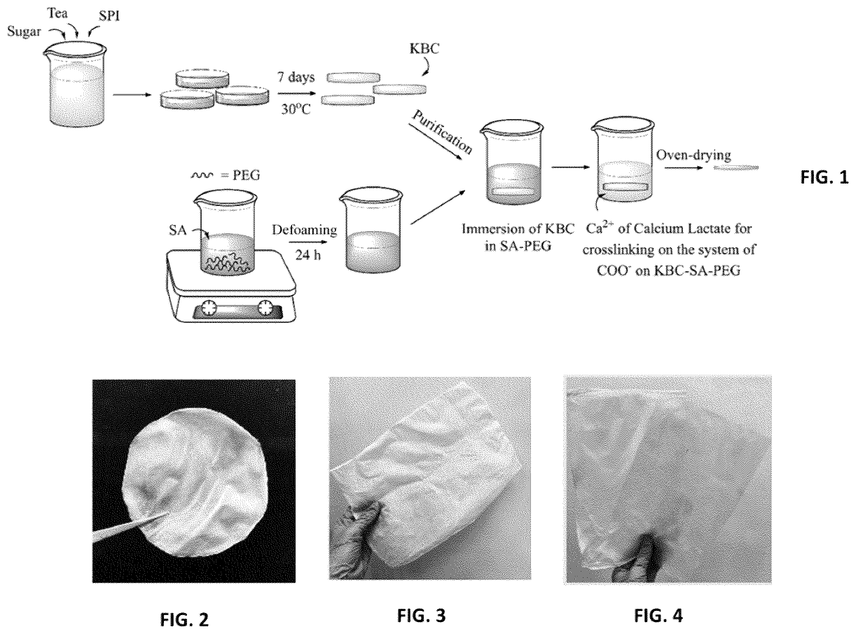

A kombucha cultivation medium with black tea powder (10.0 g/L) and sucrose (100.0 g/L) was prepared by boiling in water for 30 min with stirring, and then cooling the solution to room temperature. Kombucha SCOBY (5% per liter) was blended with a homogenizer and then added to the cultivation medium. As illustrated in FIG. 1, soy protein isolate (SPI) was added into the culture medium according to predetermined variations based on 1.0% w/v. The solution was stirred for 2 h to ensure that the SPI fully swelled, and that the culture was homogeneous. The culture was then plated in plastic containers and allowed to synthesize cellulose for 7 d at 30° C. The cultures were then washed with a 2% NaOH solution at 60° C. for 90 min and bleached with 1% NaClO at room temperature overnight to obtain a dry kombucha bacterial cellulose (KBC) film (FIGS. 2 and 3). As used in this and the following Examples and the accompanying figures, KBC refers to a film of kombucha bacterial cellulose produced using a culture medium without SPI, and KBCS refers to a film of kombucha bacterial cellulose produced using a culture medium with SPI.

### Example 2. Preparation and Application of a Coating with Calcium Alginate and Polyethylene Glycol

Sodium alginate (SA) (2% w/v, 200 mPa·s) was dispersed in deionized water at room temperature with continuous stirring (magnetic stirring at 1000 rpm) until complete dissolution. In various experiments, different molecular weights of polyethylene glycol (PEG200 and PEG400) were added into the solution as a plasticizer. The resulting solution was stirred for 4 h at room temperature and then deformed by standing at room temperature for 3 d until no gas bubbles were trapped inside. To ensure a uniform and thin alginate coating was applied on the KBCS film, the film was first immersed in the SA-PEG solution for 1 min and was then immersed in a 2% calcium lactate solution for 2 min for complete ionic crosslinking. The coated film (KBCS-Alg-PEG) was put in an oven at 110° C. for 20 min to obtain a transparent film, as illustrated in FIG. 4. Each experimental film production was carried out in triplicate.

### Example 3. Grafting of Lauryl Gallate to Chitosan Using Laccase

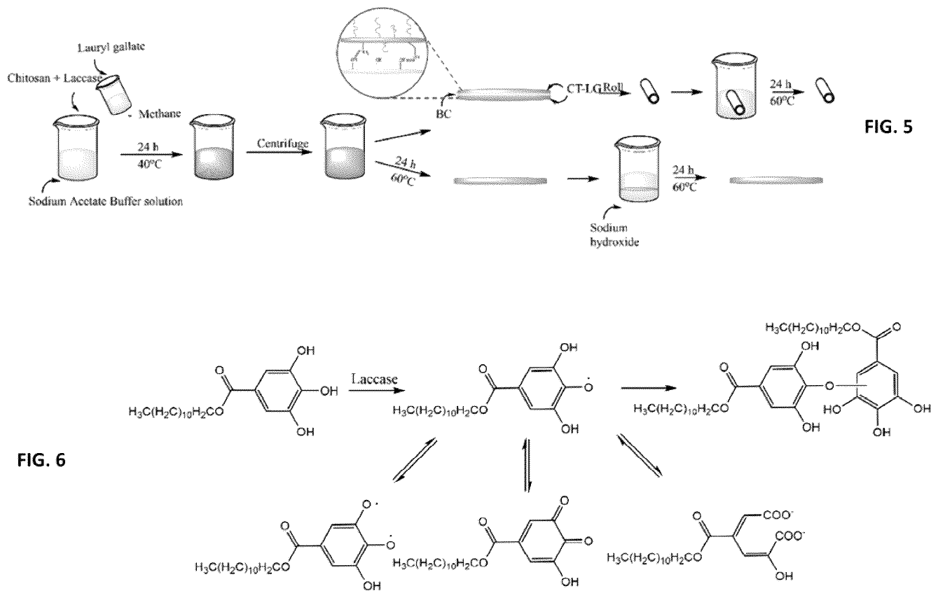

As illustrated in FIG. 5, a chitosan solution (0.2%, w/v) was prepared by dissolving chitosan powder in 20 mL sodium acetic buffer solution with the addition of 2% acetic acid under stirring overnight at room temperature. The pH of the resulting solution was 4.5. Lauryl gallate (0.2%, w/v) was dissolved in 2 mL methanol and added to the chitosan solution. 0.5 g of laccase (120 U/g) was added into the solution at 40° C. for 24 h, resulting in a satisfactorily high degree of substitution (DS) of 0.98 indicating a good reaction condition. Then, the reaction solution was centrifuged to remove unreacted lauryl gallate or lauryl gallate dimers. The resulting solution was cast at 60° C. to form a lauryl gallate grafted chitosan (CT-LG) film. This film was placed in a 0.5 M NaOH solution overnight to neutralize all the physically adsorbed acetic acid and obtain a water-insoluble CT-LG film.

### Example 4. Preparation and Application of a Coating with Lauryl Gallate Grafted Chitosan

To prepare a BC/CT-LG film as illustrated in FIG. 5, a laccase-catalyzed grafting CT-LG film was prepared by re-dissolving a CT-LG film in a small amount of acetic acid solution (10%, g/mL), and then pouring to coat a dry bacterial cellulose (BC) film (5×5 cm) surface. The coated surface was placed in a 60° C. oven for drying to obtain a BC/CT-LG composite film. The mass ratio of BC:CT-LG of the composite film was 5:1. The resulting film was placed on 0.5 M NaOH solution overnight to neutralize all the physically adsorbed acetic acid, and the film was then washed with distilled water. The resultant film was dried at 60° C. overnight.

To prepare a CT/CT-LG film, both chitosan and CT-LG (at a mass ratio of 1:1) were dissolved in a 10% acetic acid solution by stirring overnight at room temperature. The solution was then dried in an oven at 60° C. to obtain a CT/CT-LG composite film. The composite film was placed on 0.5 M NaOH solution overnight to neutralize all the physically adsorbed acetic acid, and the film was then washed with distilled water. The resultant film was dried at 60° C. overnight.

To prepare a bBC/CT-LG, blended BC (bBC), 50-100 nm in diameter and 20 m in length, was mixed with CT-LG (at a mass ratio of 1:1) in a 10% acetic acid solution by stirring overnight at room temperature. The solution was then dried in an oven at 60° C. to obtain a bBC/CT-LG composite film. The composite film was placed on 0.5 M NaOH solution overnight to neutralize all the physically adsorbed acetic acid, and the film was then washed with distilled water. The resultant film was dried at 60° C. overnight.

### Example 5. Effect of Soy Protein Isolate Concentrations on Mechanical Performance of KBCS Composite Material

Uniaxial tensile tests of films produced as described in Example 1 were performed with a tensile machine (TOHNICHI, Zhuoyue, Dongguan, China) according to the standard of GB/T 1040.3 (2006). The maximum strength in the tensile tests was 1000 N. Test velocity was selected at 4 mm/min. Sample specimens were cut according to the reference of Universal mechanical using a cutter with a mold. Testing was performed using a film gauge length of 65 mm, a temperature of 25° C., and a room humidity of 50%. Data were obtained as the averages of 3 trials. Film thickness ranges from 60 μm to 80 μm. For dry state testing, each sample was stored in a 105° C. in oven for 1 h prior to testing being performed within 15 min. The tensile strength (TS) and percent elongation at break (EAB) were each determined.

Table 1 presents the TS and EAB results for different percentages of SPI added into the culture media used to produce different exemplary bacterial cellulose films. The results demonstrate that the mechanical strength of the films varies as the SPI concentration is changed. The addition of between 0.20% and 1.00% SPI was shown to enhance the mechanical performance of the kombucha bacterial cellulose films. Without being bound to a particular theory, the inventors believe that this enhanced mechanical performance may be due to the entrapment of proteins within the bacterial cellulose matrix. Kombucha bacterial cellulose composite films produced with the addition of 0.40% SPI exhibited the greatest TS in this experiment, approximately 50% higher than films produced with no SPI addition. The films, however, tended to become more brittle with the introduction of SPI, as the EAB was reduced by as much as 72%. Balancing the impacts of SPI on both TS and EAB, the addition of 1.00% SPI achieved good results with a TS of 116 MPa and an EAB of 12.43%. The addition of greater than 1.00% SPI did not lead to further improvements in mechanical performance. Therefore, further studies were carried out using 1.00% SPI addition into KBCS as a baseline formulation.

TABLE 1
Mechanical properties of KBC and KBCS produced with
different amounts of SPI culture medium additive.

| SPI (%) | TS (MPa) | EAB (%) |
|---|---|---|
| 0.00 | 74.97 | 11.0 |
| 0.20 | 93.36 | 4.86 |
| 0.40 | 150.11 | 3.96 |
| 0.60 | 109.71 | 16.0 |
| 0.80 | 110.24 | 5.44 |
| 1.00 | 116.01 | 12.43 |
| 1.20 | 75.28 | 7.03 |
| 1.40 | 85.45 | 17.85 |
| 1.60 | 76.40 | 3.57 |
| 1.80 | 81.96 | 12.12 |
| 2.00 | 97.4 | 8.16 |

### Example 6. Effect of Soy Protein Isolate Concentrations on Growth of Bacteria and Yeast During Production of Bacterial Cellulose Fibrils

Films were produced as described in Example 1 and free bacterial cell numbers in the cultivation media were calculated. The free bacterial cell number was based on those cells that were suspended in the fermentation medium and was counted by using a plate dilution method returning values in terms of colony uniform units (CFU). From each group of cultures tested, 1 mL of tea culture broth was withdrawn from the medium on each of the first 9 days. The culture broth was diluted serially with sterile 1 phosphate buffered saline (PBS) solution. For each dilution, 100 μL of the cell suspension was spaced evenly on a 90×15 mm petri dish. These agar plates were prepared by mixing 1% of agar powder with 100 mL of boiled water. Then, following continuous boiling at 60° C. for 30 min, the slightly cooled agar was poured into the petri dish until the agar solidified. Each inoculated petri dish was inverted and incubated at 37° C. and 80% room humidity for 3 d. Colonies were then counted in each quadrant to determine the number of CFU for each cell dilution and tested condition.

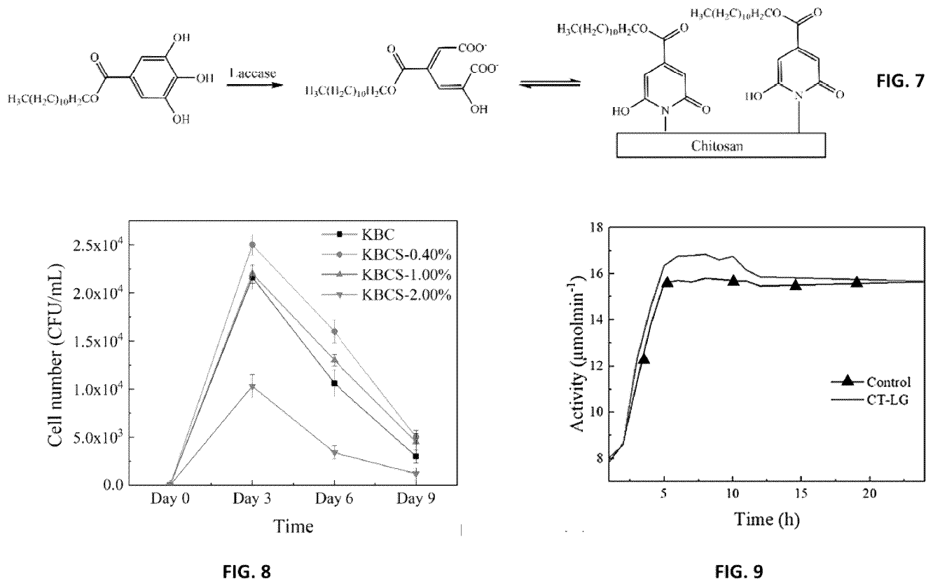

Without being bound by a particular theory, the inventors conjecture that excess SPI concentrates the solution medium resulting in a higher viscosity. This more viscous culture environment can affect the ability of bacteria and yeast to grow by constraining the availability of dissolved oxygen, the water content, and the movement of free bacterial cells. The free cell numbers of bacteria were counted in cultures with differing SPI concentrations to observe these effects of SPI on cell growth during kombucha bacterial cellulose production. As shown in FIG. 8, the addition of a low concentration of SPI coincides with observations of higher numbers of free bacterial cells, indicating a positive effect on bacterial cell production. These results suggest that SPI can provide a benefit to this complicated system as a cooperative member. The addition of high concentration of SPI, however, decreased the production of bacterial cells, possibly by inhibiting the bacterial cells' movement. The reduced activity of bacteria as high viscosity increased resistance to mass transfer for the growth of free bacterial cells eventually led to reduced BC production. Therefore, after comparing the overall mechanical property results from Example 5 and the bacterial cell production results from this Example, an addition of 1.00% SPI was selected for further investigations as a film base to receive a waterproofing coating.

### Example 7. Effect of Laccase Enzyme Activity and Degree of Reaction on Preparation of Lauryl Gallate Grafted Chitosan

Laccase activity was determined using a colorimetric assay measuring the oxidation of 2,2′-azino-bis(3-ethylbenzothiazoline-6-sulfonic acid) (ABTS). In this method, the nonphenolic dye ABTS is oxidized by laccase to the more stable and preferred state of the cation radical. The reaction solution is collected for each of the first 12 h, and then again after 24 h, to provide a total of 13 solutions to be assayed. Each of the collected solutions was mixed with 0.5 mM of ABTS substrate, 2.8 mL of sodium acetate buffer having a pH of 4.5 and a concentration of 0.1 M, and 100 μL of culture supernatant. The mixtures were then incubated for 5 minutes, and the resulting absorbance was measured at 420 nm using a spectrophotometer (UV-3600 Plus, Shimadzu, Japan) with a suitable blank. The unit of measurement for the amount of laccase was defined as the quantity oxidizing 1 μmol of ABTS substrate per minute.

FIGS. 6 and 7 illustrate the reaction mechanisms associated with entrapping lauryl gallate oligomers in the chitosan slurry, and coupling the lauryl gallate to the chitosan. The graph of FIG. 9 plots the effect of product formation on laccase activity, demonstrating that the optimized period of laccase activity was after 5 hours. Chitosan has been established as an exceptional solid support for enhancing enzyme catalytic activity and stability. Therefore, the addition of lauryl gallate and chitosan solutions resulted in a slight increase in laccase activity relative to that seen with the control group (FIG. 9).

### Example 8. Characterization of Lauryl Gallate Grafted Chitosan

Sections of CT-LG film prepared in Example 7 were placed on a specimen holder and sputter-coated with a thin layer of platinum. Scanning electron microscopy with energy dispersive X-ray spectroscopy (SEM/EDX) was then used to characterize the chemical composition of the CT-LG film. The degree of substitution (DS) of each sample, i.e., the molar ratio of the grafted lauryl gallate to the amino group of chitosan, was determined from the atomic weight percentage (awt %) of carbon in the detected region. Because chitosan has only one reaction site, the DS values ranged from 0 to 1. The results presented in Table 3 show that reaction conditions including a temperature of 40° C. and a time of 24 h resulted in a satisfactorily high DS value of 0.98.

TABLE 2
Effect of reaction parameters on lauryl gallate grafted chitosan DS.

| | | wt % | | | |
|---|---|---|---|---|---|
| | | C | N | O | DS |
| Chitosan | | 41.89 | 3.42 | 54.69 | 0 |
| Reaction | 2 | 53.17 | 6.98 | 39.85 | 0.20 |
| time | 6 | 56.95 | 7.70 | 35.35 | 0.41 |
| (h) | 24 | 62.07 | 3.27 | 34.66 | 0.98 |
| Reaction | 25 | 47.27 | 3.44 | 49.29 | 0 |
| temp. | 40 | 62.07 | 3.27 | 34.77 | 0.98 |
| (° C.) | 60 | 41.78 | 3.65 | 54.57 | 0 |

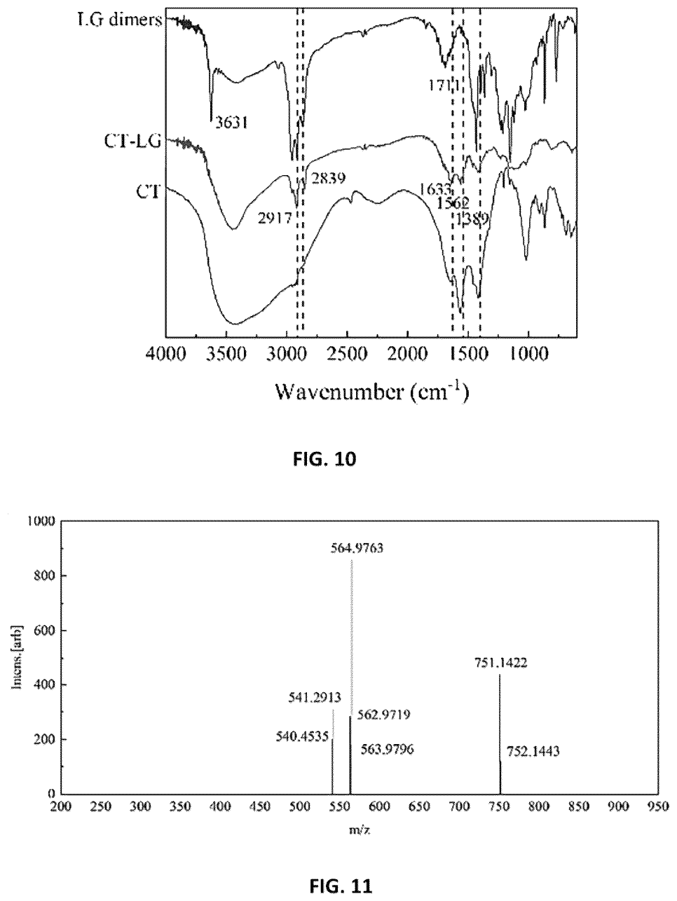

The infrared spectra of the CT-LG films were determined using a Fourier-transformed infrared analyzer (FTIR Alpha Spectrometer, Bruker, Germany). Spectra were obtained in the range of 4000-400 cm⁻¹; and an average of 24 scans at 4 cm⁻¹ resolution was taken as the final measurement. The background spectrum was collected before the test. The FTIR spectra of the synthesized CT-LG in FIG. 10 exhibits 3 identical peaks, representing CH₂ stretching at 2917 cm⁻¹, CH₃ stretching at 2839 cm⁻¹ and OH in-plane bending at 1389 cm⁻¹. These peaks indicate that the long alkyl chain, methylene, and unreacted phenol group from lauryl gallate were present on the synthesized CT-LG or CT-LG slurry. The peaks at 1562 cm⁻¹ and 1633 cm⁻¹ corresponding to the glucosamine unit had decreased absorption, which can be attributed to the bending vibrations of N—H and NH3⁺ respectively. The peak at 1711 cm⁻¹ is consistent with the effect on the carbonyl stretching band from the ester of lauryl gallate dimers.

The LG oligomer structures were confirmed by matrix-assisted laser desorption/ionization with time-of-flight (MALDI-TOF) spectrometry. MALDI-TOF spectrometry was conducted by using 2,4-dihydroxy benzoic acid as the matrix. Lauryl gallate oligomers were collected after centrifugation and dissolved in a TA30 (30% acetonitrile/70% trifluoroacetic acid) solution. The prepared solution was mixed with matrix (20 mg/mL) at a ratio of 1:1 and placed by droplet onto a ground steel plate until dry. The mass ratio (w/w) of the (CT-LG):LG dimers was measured as 4:1 after 24 h. The expected reaction mechanism for the oxidation of LG was in agreement with this MS result. Laccase removes electrons and hydrogen ions from hydroxyl groups to produce several kinds of radicals, including phenoxy radicals or quinones shown in FIG. 11. These radicals then undergo oligomerization to form lauryl gallate dimers together with an ether bond, leading to oligomers grafted onto chitosan. As shown in FIG. 11, this reaction yields products with a molecular weight distribution from 540.4535 m/z to 752.1553 m/z. Oligomerization of lauryl gallate occurs at 682.891 m/z, with the peaks at 751.1422 m/z and 752.1553 m/z indicating a different degree of oligomerization or the formation of laccase-lauryl gallate intermediate. No large molecular weights (>1000 m/z) were detected.

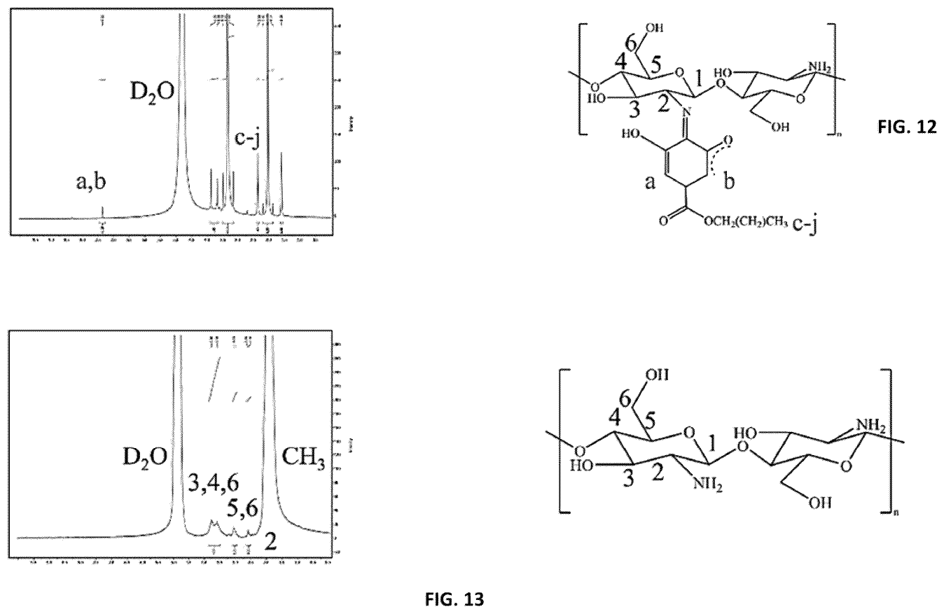

The chemical structure of CT-LG was also confirmed by ¹H-NMR analysis. ¹H-NMR samples were prepared by dissolving pure chitosan and a CT-LG film in D20 solution with a drop of acetic acid, and the samples were analyzed using an AVANCE III 400 MHz NMR Spectrometer (Bruker, Germany). FIGS. 12 and 13 present spectra of pure chitosan and CT-LG, together with corresponding structural diagrams indicating the hydrogen atoms of the spectral peaks. The alkyl chain of gallate ester is seen at 1.0-2.1 ppm (H-c-j) on the CT-LG spectrum, where such signals are missing on the pure CT spectrum. A signal of aromatic hydrogen was observed at 7.2 ppm (H-a,b). Other identifiable chitosan backbone peaks of 3-3.9 ppm are seen in both spectrums. The H-NMR results confirmed again the successful grafting of lauryl gallate onto the chitosan surface.

### Example 9. Oil Resistance Performance of Coated KBCS Composite Material

Oil penetration resistances of KBCS and KBCS-Alg-PEG200 were measured according to the TAPPI T507 method. Soybean oil was used in this test as a representative common edible vegetable oil. The films' resistance to soybean oil penetrations was evaluated and the experiment was carried out in duplicate. Films were cut with size of 5×5 cm and placed on an oil blotting paper and then an aluminum paper. Soybean oil was mixed with Nile red and dropped on the film, which was then covered with another aluminum paper. A 720-g flat-bottom bottle was placed on top of the sheet and this setup was placed in a 60° C. oven for 4 h. The oil penetration resistances of films were reported as the percentage of area exhibiting oil blots as determined using grid paper.

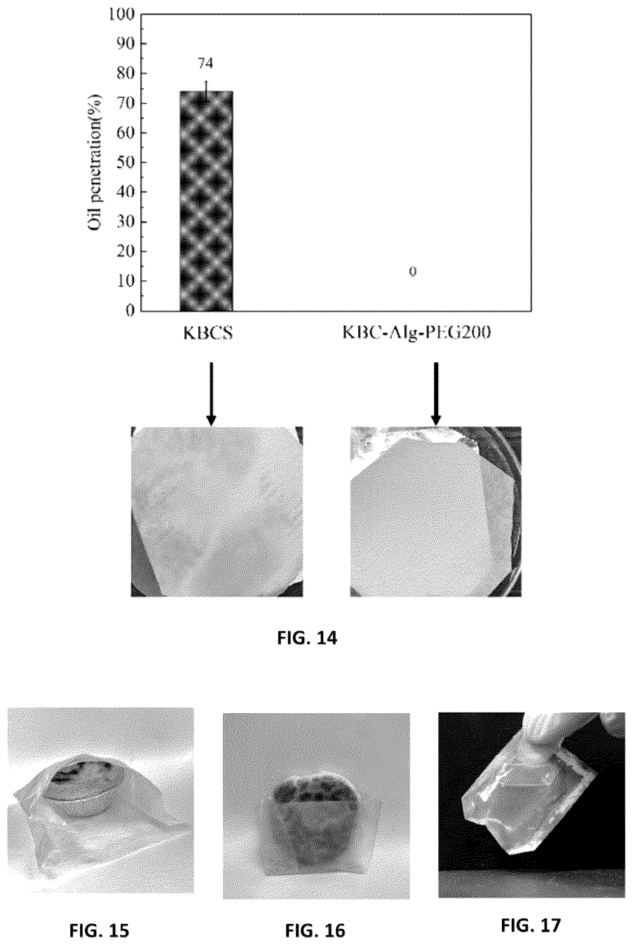

FIG. 14 presents data showing the oil resistances of the tested KBCS and KBCS-Alg-PEG200 films. The results show that with the addition of alginate/alginate-PEG coating, the provided film can completely block oil penetration, resulting in a completely clean blotting paper. In contrast, the uncoated bacterial cellulose exhibited weak oil resistance, likely related to the porosity of the cellulose surface and the large pore size of the cellulose matrix. The hydrophilic characters of alginate and the ability of alginate to penetrate the BC matrix and fill these pores significantly and advantageously improved this characteristic of the composite material. Because the oil barrier property of a material is often an essential consideration in food packaging industries, the provided coated composite film with high oil resistance can be suitably used for food packaging, even for the packaging of oily and crispy snacks (FIGS. 15-17).

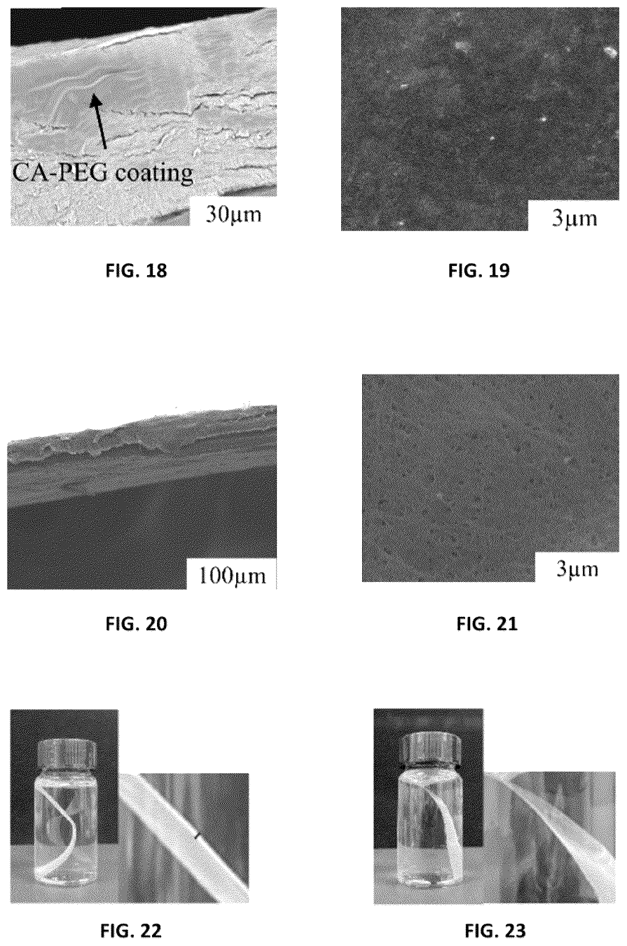

The surface morphology of the samples was also captured using scanning electron microscopy (SEM). Samples were placed on a metal plate and attached with conductive tape. The SEM (JSM-7800F, JEOL, Tokyo) was operated at 1,000-50,000× magnification with a 10.0-kV electron beam and a working distance of about 10 mm. Imaging was conducted with a backscattered electron detector for both cross-section and surface. The SEM images of FIGS. 10 and 11 show that the additional alginate or alginate-PEG coating allows the SA to penetrate the pores in the 3D nanoscale network of BC films. The surface of KBCS fibrils covered with alginate-PEG (FIG. 18) was much smoother than the rough surface of uncoated KBCS (FIGS. 20 and 21), with no pores observed in the coated film.

### Example 10. Low Hydroscopicity Performance of Composite Materials

The water barrier properties of films, such as moisture content, water sorption, and water permeability, can play a critical role in evaluating the applicability of the films for use in food packaging. Since alginate is typically used in the form of a hydrogel with a three-dimensionally cross-linked network composed of hydrophilic polymers with high water content, it is well known that alginate absorbs high amounts of water. This ability of alginate arises from hydrophilic functional groups of the polysaccharide attached to its polymeric backbone. Therefore, to increase the resistance of the alginate containing KBCS films to swelling, a polyether such as polyethylene glycol can be used, where the sizing of the polyether is tuned to the smaller pores of the alginate matrix.

The moisture content (% MC) of KBCS films or alginate coatings was measured by first weighing and recording their initial weights (W₁) at 50% humidity and room temperature, and then recording their weights after oven drying at 105° C. until a constant value (W₂) was reached. The percentage of moisture absorbed by the film or alginate coating was calculated using the equation:

$$\% M C = ( {W}_{1} - {W}_{2} ) / {W}_{1} \times 100 \%$$

Each dry sample from the water content test was soaked with water in a small flask for 24 h, and then weighed to determine a wet sample weight (W₃). The water absorption (% WA) of the sample was calculated to determine the swelling ability of the sample using the equation:

$$\% W A = ( {W}_{3} - {W}_{1} ) / {W}_{1} \times 100 \%$$

A water drop test (WDT) performance metric was measured for the samples by recording the time needed for complete absorption of a drop of water (5 μL).

The water vapor transmission rate (WVTR) of samples was measured by using a gravimetric method. Samples were maintained at 75% room humidity and 30° C. in a test chamber (SUNNE, Shangpu Instrument Equipment, Shanghai, China). A cylinder (diameter of 10 mm) was filled with 5.0 g calcium chloride anhydride granules. The cylinder was covered with the sample to be measured, and the weight increase of the sample after 24 h was recorded. The WVTR of the samples was measured using the equation:

$$W V T R = \frac{Δ mass}{{r}^{2} π \times time}$$

The water contact angle (WCA) of samples was measured using a contact angle tester (OCA 25, Dataphysics Company, Germany) with a droplet method using SCA_25 software with sessile drop mode. Images were captured with 2 μL deionized water applied for 30 min.

The data of Table 3 presents results from tests of the water barrier properties of KBCS composite films including polyethylene glycol of different molecular weights. The results show that PEG200 provided particularly good performance in all water resistance tests, demonstrating that PEG200 can have a synergistic effect as a binder between alginate and KBCS, as well as acting as a plasticizer to enhance water resistance.

TABLE 3
Water resistant analysis of KBC, KBCS, and KBCS with alginate or alginate-PEG coatings.

| | | | | KBCS-Alg- | KBCS-Alg- |
|---|---|---|---|---|---|
| | KBC | KBCS | KBCS-Alg | PEG200 | PEG400 |
| MC (%) | 28.55 ± 1.39 | 24.83 ± 3.48 | 17.55 ± 2.19 | 10.23 ± 1.19 | 16.49 ± 1.37 |
| WA (%) | 244 ± 10.0 | 261.68 ± 19.9 | 121.37 ± 13.9 | 82.12 ± 13.44 | 122 ± 17.23 |
| WVTR (g/(m² · hr)) | 172.20 ± 11.0 | 211.86 ± 2.56 | 117.68 ± 0.83 | 107.36 ± 9.91 | 181.96 ± 5.64 |
| WCA (degrees) | 12.67 ± 0.73 | 58.55 ± 0.78 | 12.70 ± 4.22 | 104.2 ± 2.72 | 77.46 ± 0.26 |

As shown in Table 3, the water absorption properties of films with alginate or alginate-PEG coating (82.12±13.44 to 122±7.55) differed from that of films without coating (244±10.0 to 261.68±19.9). For the uncoated films, the high porosity and the dense three-dimensional networks of KBC lead to an excellent water swelling capacity. Since the molecules of an alginate or alginate-PEG coating can penetrate the BC matrix and fill the pores, less water was absorbed in films with these coatings after 24 h. Moreover, the water absorption decreased further with coatings containing added PEG in comparison to coatings with pure alginate. Without being bound to a particular theory, the inventors believe this observation can be explained by hydrogen bond interactions between the accessible carboxyl groups and hydroxyl groups of alginates and the hydroxyl groups of PE. The formation of these hydrogen bonds may allow the PEG plasticized films to contain less sorption sites for water than the original alginate film. Images of KBCS (FIG. 22) and KBCS-Alg-PEG200 (FIG. 23) immersed in water after 30 min also show that the alginate-PEG200 coating can protect the KBCS film from water and allow the film to avoid swelling behavior. The PEG200 thus effectively acts as binder and plasticizer, packing the coating with calcium ions through strong electrostatic bonding with alginate.

The water vapor transfer rates of all films coated with alginate or alginate-PEG decreased relative to the rates observed for original KBC and KBCS films. The KBCS-Alg-PEG200 film exhibited the lowest WVTR (107.36±9.91), representing a 37.8% decrease relative to the uncoated KBC film without SPI (172.20±11.0). This result was consistent with that seen for the water absorption and moisture content. However, the overall WVTR values of all alginate or alginate-PEG coated films were high because of the high-water affinity and high permeability of the alginate hydrogel.

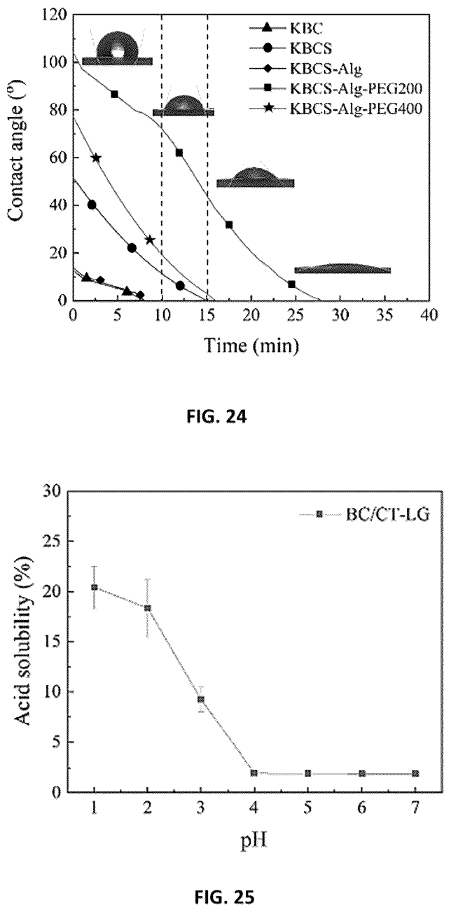

Water contact angle measurements (FIG. 24) provided additional information about the surface hydrophobicity values for the tested films. The PEG200 coated film in particular exhibited a significantly increased water contact angle. This result indicates that an increased hydrophobic property is associated with the PEG200 carbon chain.

The results from water contact angle (WCA), water vapor permeability (WVP), and water drop test (WDT) assays also confirmed that applying an enzymatic treatment to graft lauryl gallate directly to chitosan successful produces a hydrophobic material. Moreover, the material is advantageously able to maintain its hydrophobic character when used as a coating on a bacterial cellulose surface.

Table 4 presents results from assessments of the hydrophobicity of a BC/CT-LG composite film. The WDT data in the table show that the initial absorption times for chitosan and BC were 2 min 16 s±7 s and 5 min 35 s±12 s, respectively. After grafting of lauryl gallate to the chitosan material, water droplets needed more than 15 min for complete absorption. For BC treated with CT-LG coating, the same result was shown, with more than 15 min required for complete absorption. In contrast, control groups of CT or BC film mixed with CT-LG and lacking completely covered surfaces exhibited no dramatic increase in absorption time, which was consistent with the result in WCA measurement.

TABLE 4
Water resistant analysis of CT, BC, CT-LG,
CT/CT-LG, bBC/CT-LG and BC/CT-LG films.

| | Water Barrier Properties | | Hydrophobicity |
|---|---|---|---|
| Specimens | WDT (time) | WVTR (g⁻²h⁻¹) | WCA (°) |
| CT | 2 min 16 s | 131.30 ± 13.8 | 52 ± 0.54 |
| BC | 5 min 35 s | 172.20 ± 11.0 | 12.67 ± 0.73 |
| CT-LG | >15 min | 84.33 ± 7.3 | 120 ± 0.40 |
| CT/CT-LG | 7 min 57 s | 131.53 ± 6.4 | 55 ± 0.94 |
| bBC/CT-LG | 6 min 46 s | 159.17 ± 9.8 | 72.2 ± 0.45 |
| BC/CT-LG | >15 min | 83.23 ± 5.3 | 120 ± 0.96 |

As also seen in Table 4, the WVTR and WVP of BC/CT-LG decreased as compared to that of the BC films. CT-LG had the lowest WVP (0.982±0.202 gμmm⁻² day⁻¹ Pa⁻¹), which represented a 600% decrease compared with that of pure CT film (6.061±0.287 gμmm⁻² day⁻¹ Pa⁻¹). Furthermore, the WVP of BC/CT-LG (1.153±0.031 gμmm⁻² day⁻¹ Pa⁻¹) decreased 55% from that of the original BC film (2.563±0.16 gμmm⁻² day⁻¹ Pa⁻¹). These results were consistent with those of the WDT and WCA analyses, and provided further evidence of the beneficial hydrophobic properties and low porosity of CT-LG.

The initial WCA values of CT and BC were 52±0.54° and 12.67±0.73° respectively, indicating typically hydrophilic surfaces. Following the enzymatic modification of chitosan to prepare CT-LG, the WCA of the prepared material increased to 120±0.40°, showing the surface of the material became hydrophobic. Without being bound by a particular theory, the inventors believe that the enhanced hydrophobicity of CT-LG results from the long lauryl gallate hydrocarbon chain added to the surface of the chitosan. Moreover, for the BC/CT-LG composite material, the addition of the CT-LG coating to the BC surface led to a higher contact angle of 120±0.96°, consistent with the hydrophobic character of the coating. In contrast, the control composite material formed by mixing CT with CT-LG exhibited no dramatic increase in hydrophobicity, which can be attributed to its lack of a completely covered surface. This result indicated that complete surface coating, rather than blending, is needed for the CT-LG material to provide waterproofing properties.

### Example 11. Acid Solubility Performance of BC/CT-LG Composite Materials

An acid solubility test was performed on the BC/CT-LG film material to evaluate its suitability for use with acidic drinks or food. A BC/CT-LG composite material was dried in an oven for 24 h at 105° C., while solutions having different pH values were prepared by addition of HCl. Approximately 0.05 g of BC/CT-LG was then placed in a beaker with 10 mL of a prepared pH solution. The solution was then stirred for 24 h and washed with deionized water. The residual film was dried again at 105° C. for 24 h and weighed. The acid solubility in different pH of the BC/CT-LG films was determined using the following formula, in which W₃ and W₄ are the weights of the dry film before and after immersion in the acidic solution, respectively.

$$Acid solubility ( \% ) = \frac{{W}_{3} - {W}_{4}}{{W}_{3}} \times 100 \%$$

FIG. 25 presents data measured after subjecting BC/CT-LG films to various pH levels ranging from pH=1-7. The results in the graph show that only those materials exposed to solutions at pH=1-2 exhibited a high dissolution ratio, ranging from 20.42±4.25% to 18.34±5.86%, indicating a high acid solubility of the CT-LG coating solution under these very low pH conditions. The dissolution ratio reduced from 18.34±5.86% to less than 10% at pH=3-4.

At higher pH levels, the films remained substantially intact, suggesting that they would be suitable for use with a wide range of weakly acidic environment, such as those commonly encountered in the food and beverage industry.

### Example 12. Dry and Wet State Mechanical Properties of Composite Materials

Uniaxial tensile tests of films were performed with a tensile machine (TOHNICHI, Zhuoyue, Dongguan, China) according to the standard of GB/T 1040.3 (2006). The maximum strength of the tests was 1000 N. Test velocity was selected at 4 mm/min. Sample specimens were cut according to the reference of Universal mechanical using a cutter with a mold. The film gauge length was 65 mm, and the test was performed at 25° C. and 50% humidity. Reported data represents the averages of 3 trials. The film thickness for Alg-PEG coated KBC ranged from 60 μm to 80 μm. The thicknesses of CT and CT coated BC film ranged from 20 μm to 40 μm, and from 0.5 mm to 1.0 mm, respectively. For dry state tests, samples were stored at 105° C. in an oven for 1 h and the test was then performed within 15 min. For wet state tests, samples were completely immersed in deionized water for 15 min before testing. Both the TS and EAB were calculated for the wet state and dry state samples.

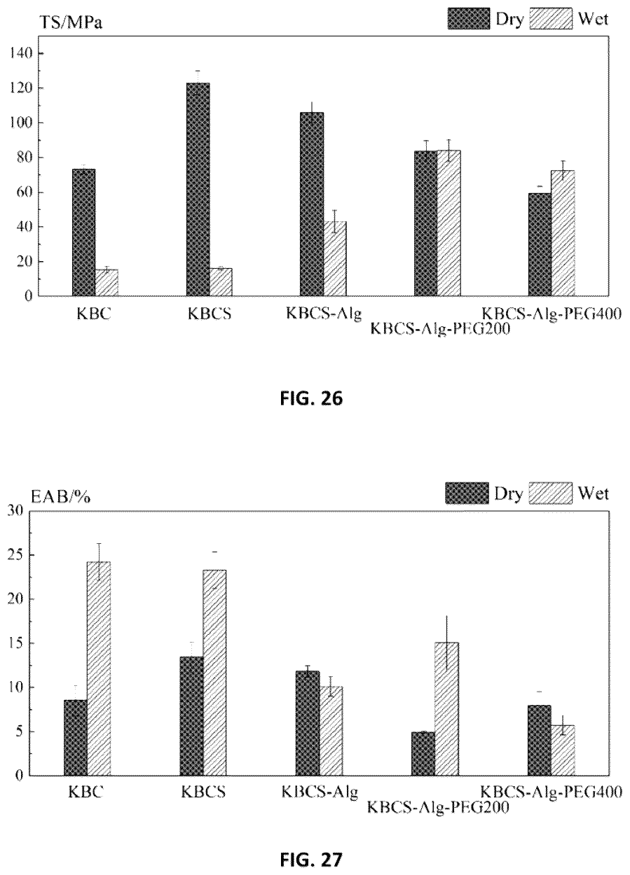

FIGS. 26 and 27 presents the TS and EAB results, respectively, of dry and wet state tests of KBC, KBCS, KBCS-Alg and KBCS-Alg-PEG films. The TS of in-situ modified KBCS was measured as being 70% higher (123±6.9 MPa) than that of unmodified KBC (73.2±2.7 MPa), whereas the EAB of the KBCS film was 57% higher (13.4±1.7%) than that of the KBC film (8.5±1.8%) in a dry state. After a 15 min immersion in water, both KBC (15.3±2.0 MPa) and KBCS (16.3±1.0 MPa) swelled and loosened due to the high water-holding capacity of bacterial cellulose. This finding was in good agreement with previous research and demonstrated that the highly hydrophilic and humectant nature of bacterial cellulose is characterized by an elevated water uptake capacity. The addition of an alginate coating yielding KBCS-Alg caused the film to exhibit a slightly lower TS (106±6.4 MPa) in dry state, while doubling the TS value (43.1±6.5 MPa) seen in wet state. This result was likely due to high-water permeability allowing for the loosening of the KBC fibrils.

The addition of PEG further weakens the TS in dry state due to its plasticizer effect, such that KBCS-Alg-PEG400 showed a greater decrease of 51.8% (59.3±4.1 MPa) relative to that of the KBCS film. Both KBCS-Alg-PEG200 and KBCS-Alg-PEG400 exhibited a better TS in wet state (72.6±5.7 MPa) than dry state (59.3±4.1 MPa). This observation was likely due to water molecules penetrating the KBC network and forming a more rigid structure with packed and compact hydrogen bonding. The water molecules can thus act as a plasticizer agent in fiber-reinforced composites, leading to an increase in material ductility.

Of the tested films, KBCS-Alg-PEG200 had the highest and most stable TS performance in both dry state (83.6±6.2 MPa) and wet state (84.0±6.4 MPa). Without being bound by a particular theory, the inventors believe that that an interpenetrated network of KBC, SPI, and alginate fits particularly well with the lower molecular weight of PEG200. In particular, the low molecular weight of the PEG200 polymer may favor polymer chain mobility and avoid bridging flocculation when combined with the alginate polysaccharide. The PEG200 thus may create more intermolecular interactions and achieve a relatively high-water resistance by binding the composite material together and preventing the film from excessively swelling (FIG. 15). The difference between observed TS behaviors of the PEG200 and PEG400 films suggests that the size of these molecules impacts their abilities to penetrate internal pores of the composite film. A shorter molecule chain (PEG200) with a smaller size may more easily penetrate the small pores of KBCS in a collaborative network of KBCS and alginate films.

Both KBC and KBCS exhibited high EAB values in wet state, likely due to the strong plasticizer effect of water. Without the waterproofing alginate coating, the plasticizing activity of water may weaken hydrogen bonds and dipole-dipole intra- and inter-macromolecular interactions by shielding of these mainly attractive forces, resulting in lower stress and increased strain. The EAB did not differ much between the alginate and alginate-PEG coated films in both wet and dry state. The alginate presumably packed closely with the KBC fibrils and limited the water absorption, further reducing the elasticity of the network and resulting in a less stretchable and flexible structure.

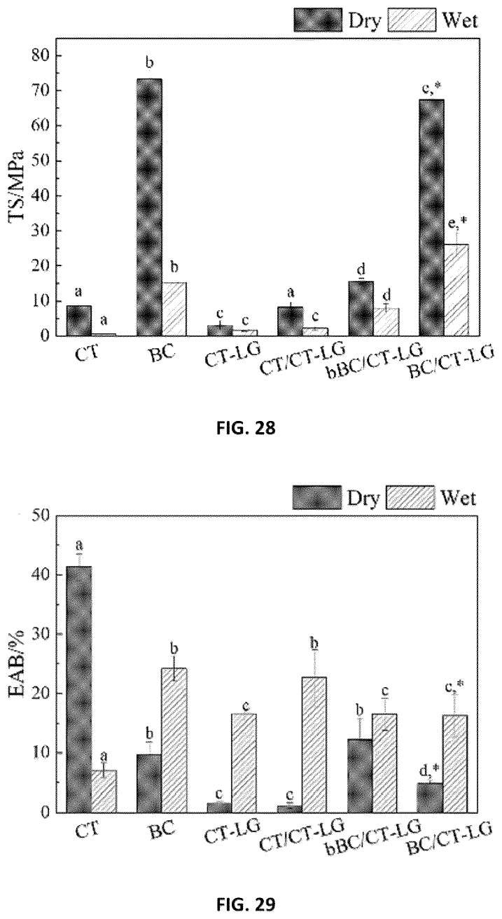

As shown in FIG. 28, the dry state and wet state tensile strengths (TS) of the original chitosan material were only 8.49±1.51 MPa and 0.668±0.12 MPa respectively, indicating poor mechanical strength and poor water stability. The grafted CT-LG films exhibited a lower dry TS (3.11±1.47) but a higher and more stable wet TS (2.20±0.67), indicating greater water stability than that of the original film. Still, the mechanical strength of CT-LG films was insufficient for the material to be suitable for use in forming, e.g., a drinking straw. By combining this material with BC, the resulting wet BC/CT-LG films retained 38.5% of the dry strength, whereas wet BC retained only 20.9% of its dry strength. Further, the wet state TS of BC/CT-LG improved 69.7% from that of the original BC film. The wet TS value for BC/CT-LG films (26.0±3.45 MPa) is not only remarkably superior to that of Kraft paper (˜4.6 MPa), but also much higher than commercial plastics, like LDPE (˜11 MPa). Accordingly, the new material has a significant potential as an alternate substitute for plastics. Without being bound by a particular theory, the inventors believe that the blending process in the formation of bBC/CT-LG disrupts the highly ordered structure of BC films, resulting in a less ordered structure that is more prone to deformation and failure under stress, and leading to the lower dry TS shown in FIG. 28. In its wet state, the bBC/CT-LG film (8.01±1.25 MPa) retained 38.5% of its dry strength (15.64±0.82 MPa).

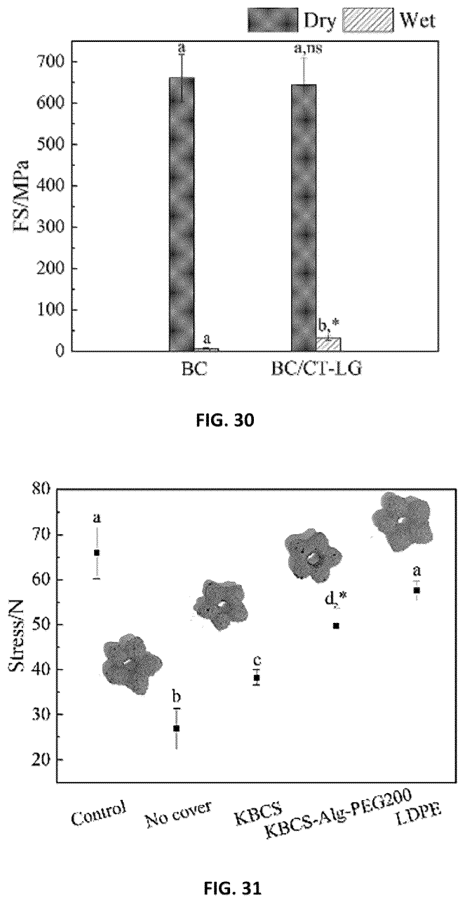

The dry state and wet state flexural strengths of BC and BC/CT-LG were also measured with these materials having the shape of a drinking straw. The data presented in FIG. 30 were obtained by measuring the performance of these shaped materials under a comprehensive force. The wet FS and toughness of BC/CT-LG significantly outperformed those of a straw made from pure BC, which exhibited substantial softening in water. Without being bound by a particular theory, the inventors believe that this improvement of wet FS may be attributed to the CT-LG uniformly covering the surface of BC film and forming a hydrophobic layer which contributed to the water repelling. Both BC and BC/CT-LG showed tough and strong FS in a dry state, with measured values of 660.1±57.64 MPa and 645.2±64.58 MPa respectively. The wet FS of BC and BC/CT-LG were measured at 6.47±2.11 MPa and 32.45±6.52 MPa respectively, with modified BC straws reaching a strength four-fold higher than the wet FS of original BC straws. Because the wet FS of paper and plastic drinking straws have been reported as 11.7±1.1 MPa and 12.5±0.1 MPa, respectively, the BC/CT-LG (32.45±6.52 MPa) material clearly outperforms commercially available counterparts. Together, these results demonstrate that the provided CT-LG coating can improved the TS and FS of BC in a watery environment, resulting in a stronger and much stable material. This waterproofing effect is attributed primarily to the hydrophobic characteristics of lauryl gallate grafted on the chitosan surface.

### Example 13. Use of the Provided Composite KBCS-Alg-PEG200 and BC/CT-LG Materials in Water-Resistant Packaning Bags and Drinking Straws

Enhancing moisture or gas barriers is one of the most significant properties of bioplastics materials. Water vapor permeability tests were performed to show that an enhanced moisture barrier is maintained with the provided KBCS-Alg-PEG200 and BC/CT-LG films. Additionally, cereal from HEYROO (Korea) was used to test crispness after a week storage in KBCS-Alg-PEG200 bags under 60% relative humidity and 25° C. Other experiments tested the use of the BC/CT-LG bag to contain ketchup.

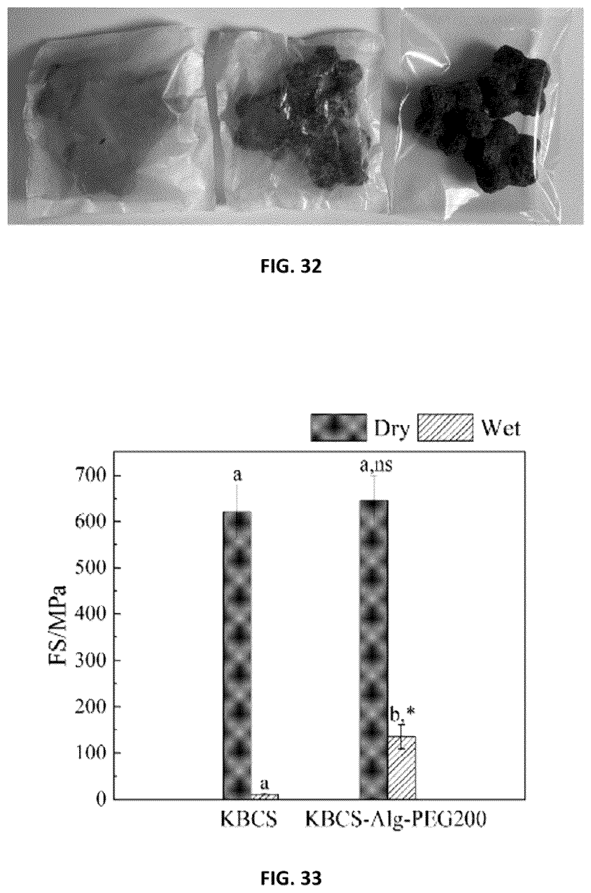

Results demonstrated that KBCS-Alg-PEG200 films were effective in retaining the crispness of oily cereal as shown in FIG. 31. The maximum stress for compression decreased 59.2% in the time between the first day (65.9±5.66 N) and the seventh day with no cover (26.9±4.53 N), indicating that the cereal absorbed moisture in the environment and became loosened. Decreases observed when using KBCS-Alg-PEG200 (49.7±4.04 N) or KBCS (38.3±1.72 N) were 24.6% and 41.9%, respectively, indicating that KBCS-Alg-PEG200 performed better than KBCS. A reference sample using commercial plastic LDPE (57.6±2.15 N) showed no significant change in the crispness of cereal, due to the high moisture barrier provided by the plastic. All 4 samples had no appearance difference. While KBCS-Alg-PEG200 thus had a slight drop in compression stress, it was the most comparable film to LDPE among all the groups. As a result, KBCS-Alg-PEG200 was found suitable for producing a single-use bag, illustrated in FIG. 32. This provided food container can be used for snacks, candy, street food, bread, or any other goods with similar properties.

Hydrostability is a critical factor for straws used for drinking. To test the hydrostability of KBCS film with Alg-PEG200 coating, the coated film was formed into a straw shape by rolling with a polytetrafluoroethylene rod and then immersing the rolled film in a calcium lactate solution overnight to allow interpenetration of Ca²⁺ ions layer-by-layer. Also, BC/CT-LG composite films were rolled into a drinking straw shape with the help of a polytetrafluoroethylene rod and then dried in a 60° C. oven before neutralizing with acetic acid.

The flexural strength (FS/MPa) of the KBCS-Alg-PEG200 drinking straws was measured, providing the data plotted in FIG. 33. The results show a comparison of the magnitude of force required to bend the straws in different conditions. The flexural strength of KBCS-Alg-PEG200 (645.2±54.82 MPa) reduced 79% from its dry state due to the hydrophilic nature of cellulose and alginate, and capillary effects in cellulose fibers. However, KBCS-Alg-PEG200 had a 13-fold higher wet FS (135.46±26.52 MPa) than the KBCS (10.74±2.31 MPa), as well as an FS value significantly higher than that of commercial paper straws (˜4 MPa), indicating a better water-resistance.

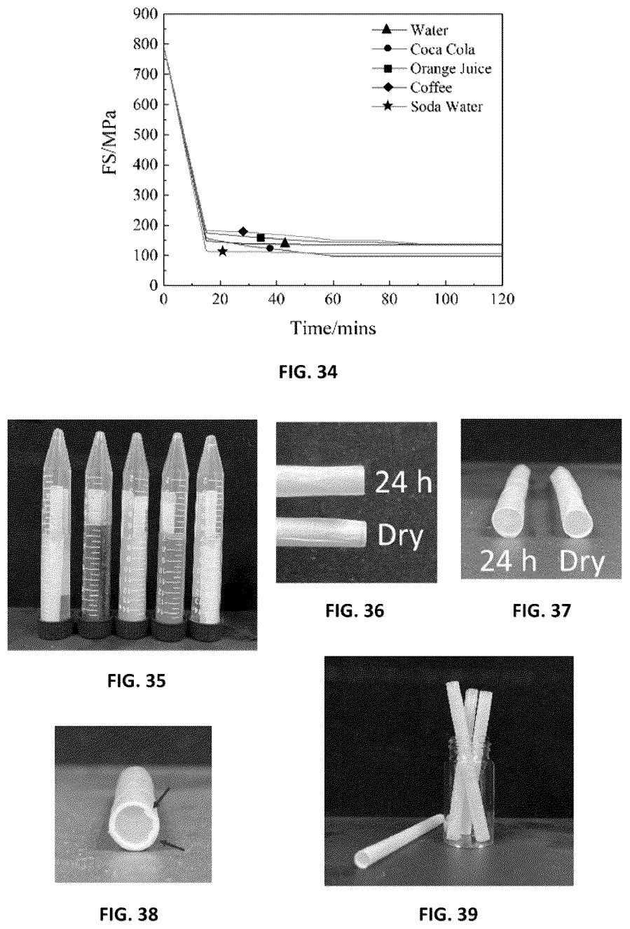

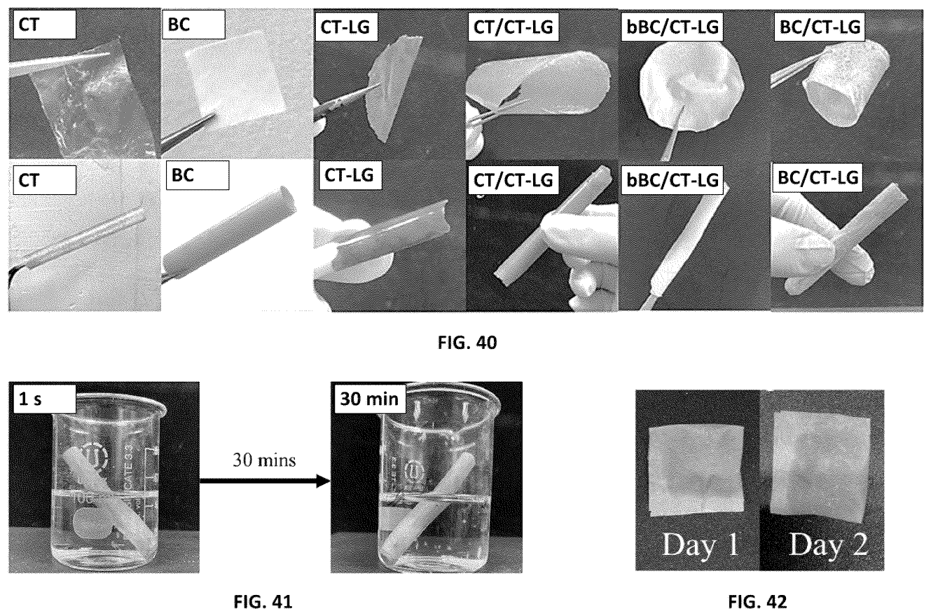

Hydrostability tests of straw samples were also conducted in neutral water, acidic drinks (cola, orange juice), and alkaline drinks (coffee, soda water), with results presented in FIG. 34. A clear drop of FS was observed in all samples within 15 min of immersion, and then stable performance was maintained for the following 2 h of immersion, indicating good durability in the various solutions. Samples immersed in cola (˜97.5 MPa) and soda water (˜108 MPa) had a slightly lower FS than other samples. This was likely due to the formation of carbon dioxide bubbles that accelerated the penetration of water molecules into the straws. Other samples, such as those immersed in orange juice (˜137 MPa) and coffee (˜140 MPa) exhibited a stable performance that was comparable to the control sample immersed in water. A commercial paper straw immersed in water for 24 h was used as a reference. FIG. 38 shows that the cellulose fiber swelled and formed an uneven surface in this reference sample because of the hydrophilic nature of cellulose. Since KBCS-Alg-PEG200 is characterized by a lower water absorption, no such issues appeared on the provided straws. FIGS. 36 and 37 show that these straws experienced no substantial elongation on their front or side surfaces, with no apparent swelling detected. Accordingly, the provided straws prepared using the materials disclosed herein can maintain a smooth surface and structural integrity. Moreover, the hardness of the KBCS-Alg-PEG200 drinking straws in the dry state (645.2±54.82 MPa) enables it to, for example, pierce a plastic membrane of a beverage in a commercial bubble tea shop. These results therefore demonstrate that the hydrostability of KBCS-Alg-PEG200 provides an advantage over commercial paper straws.

FIG. 41 shows that, following immersion in water for 30 min, a straw made with BC/CT-LG showed no peeling of its coating. Relatedly, the drinking straws shape of this structure can be maintained following the extended immersion.

FIG. 42 shows a test of ketchup storage in BC/CT-LG bags over the course of 24 h. While there was a ˜30% decrease in weight due to the loss of water vapor, no ketchup leakage occurred with these containers.

### Example 14. Transparency Properties of Provided Composite Materials

The transparency of the provided films was analyzed by measuring the transmittance of the films between the wavelengths 200 nm and 800 nm using a spectrophotometer (UV-3600 Plus, Shimadzu, Japan). The transparency of films at 600 nm was calculated according to the equation:

$$Transparency = ( Log \% {T}_{600} ) / x \times 100 \%$$

where % T600 is the percent transmittance at 600 nm and x is the film thickness (mm).

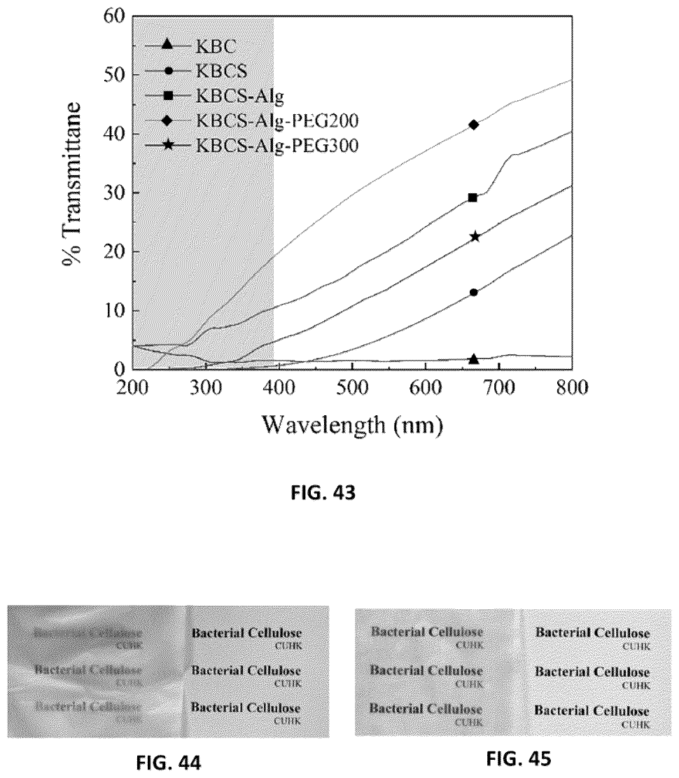

FIG. 43 and Table 5 present transmittance data for various provided films in the UV-vis region (200-800 nm). Higher values of transparency are indicative of a more transparent film. Note that for the unmodified and uncoated KBC film, the tea medium was comparable to the HS medium, and the brown color pigment of KBC can be successfully washed during film purification steps. All the alginate or alginate-PEG coated samples showed an increased transmittance of the films (17.86 to 26.15) relative to that of the KBC sample (2.88) and the KBCS sample (16.1). The KBCS-Alg-PEG200 film exhibited the highest percent transmittance (37.09%) at 600 nm. Without being bound by a particular theory, the inventors believe that alginate and PEG200 may facilitate the retention of a homogenous structure in KBCS, thereby improving its transparency. As seen in FIGS. 44 and 45, the printed text of “Bacterial Cellulose CUHK” can be completely and easily viewed through the KBCS-Alg-PEG200 film, but not through the KBCS film. These results demonstrate that satisfactory transparency, e.g., transparency comparable to that of petroleum-based plastics commonly used for food packaging, can be achieved with the addition of a polyether such as PEG200 to the composite material coating.

TABLE 5
Transparency calculation values for KBC, KBCS, and
KBCS with alginate or alginate-PEG coatings.

| | | | | KBCS-Alg- |
|---|---|---|---|---|
| | KBC | KBCS | KBCS-Alg | PEG200 |
| T₆₀₀ | 0.0156 | 0.0859 | 0.249 | 0.371 |
| % T₆₀₀ | 1.56 | 8.59 | 24.89 | 37.09 |
| Transparency | 2.88 | 16.1 | 22.16 | 26.15 |

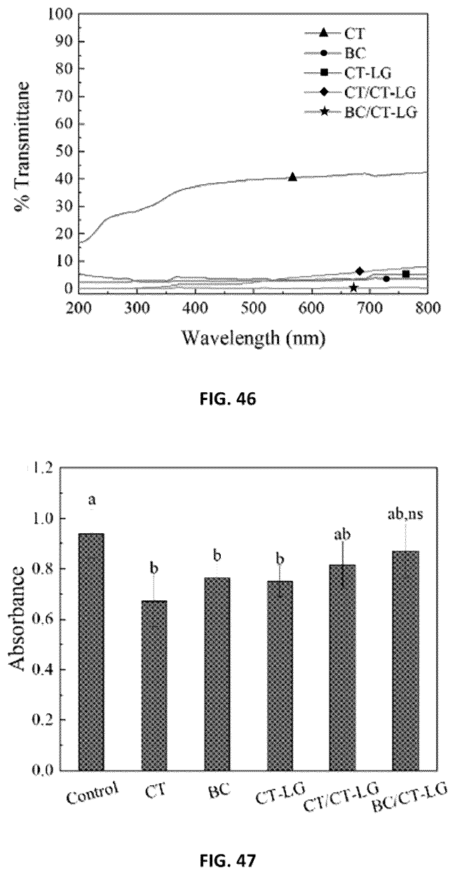

Pure chitosan film exhibits a very high transmittance of UV light, indicating a weak UV blocking property. This is most likely due to chitosan having a relatively amorphous structure lacking a regular arrangement of molecules that can effectively absorb or scatter UV radiation. After grafting chitosan with lauryl gallate, the transmittance of UV light decreased significantly by about 90.0%-93.0%, as shown in the data of FIG. 46 and Table 6. Without being bound by a particular theory, the inventors believe that the increased UV blocking property of the CT-LG composite film is primarily due to the phenol moieties of lauryl gallate. Pure BC film showed a high UV blocking properties due to presence of certain functional groups in the cellulose structure of this material, as well as its high degree of crystallinity. For BC/CT-LG, since both BC and CT-LG have high UV blocking properties, the composite material exhibited almost 100% UV-A, B, and C protection. However, of these materials, only the chitosan film had a high transparency to visible light.

TABLE 6
Optical properties of CT, BC,
CT-LG, CT/CT-LG, and BC/CT-LG.

| | UV-A | UV-B | UV-C | % T600 | Transparency |
|---|---|---|---|---|---|
| CT | 25.65 | 32.56 | 39.46 | 40.86 | 80.56 |
| BC | 1.834 | 0.153 | 3.277 | 1.56 | 2.88 |
| CT-LG | 2.560 | 2.714 | 2.769 | 3.195 | 12.61 |
| CT/CT-LG | 0.107 | 0.108 | 0.108 | 2.531 | 1.08 |
| BC/CT-LG | 0.107 | 0.108 | 0.108 | 0.280 | 0.00 |

### Example 15. Non-Toxicity of the Provided Composite BC/CT-LG Film

The cytotoxicity of the provided composite BC/CT-LG film was measured with the MTT Assay using the Leaching liquor method. A cell suspension (at a density of 1×10⁴ cells/mL) was incubated in microtiter 96-well plates for 24 h, both with and without 5 mg of the composite BC/CT-LG film. Afterwards, the MTT reagent (5 mg/mL) was added to the plates, which were then incubated for an additional 4 h at 37° C. After incubation, all media was replaced with 200 μl of a 99.5% DMSO solution. The absorbance (OD) of the solution was measured at 540 nm using a microplate reader (Labexim Products LEDETECT 96, EU). Cytotoxicity as represented by cell survival was calculated using the following formula.

$$Cell survival = Cell viability ( \% ) = \frac{Absorbance of sample}{Absorbance of control group} \times 100 \%$$

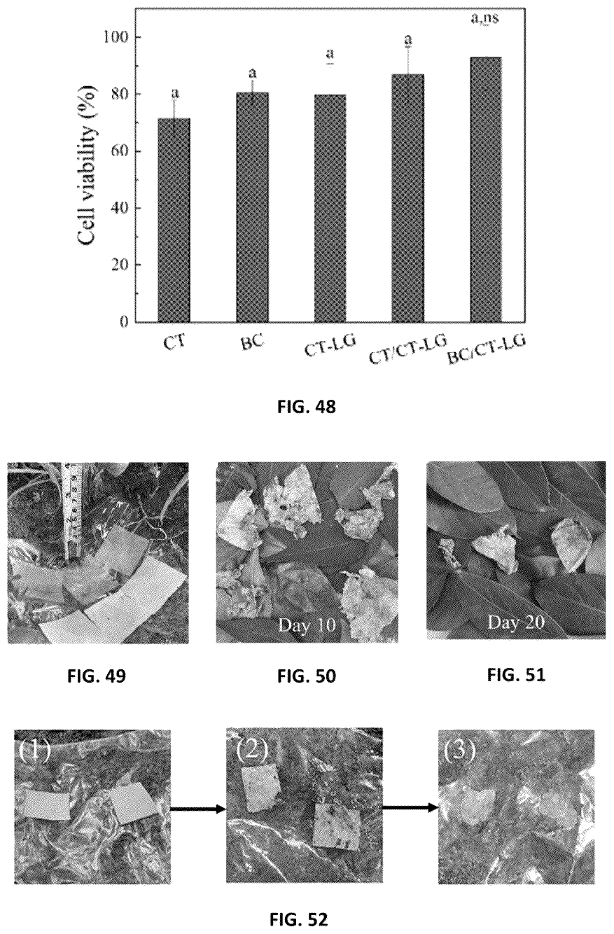

FIGS. 47 and 48 show absorbance and cell viability data from the cytotoxicity tests. The data of FIG. 48 demonstrate that the cell viability in all samples was satisfactory, with measured values ranging between 80%-90%. These results indicate that the cell cultures were not adversely affected by the presence of films. Further, no significant reduction in the viability was found when comparing CT-LG, CT/CT-LG, and BC/CT-LG sample with a control group. This finding provides evidence that the synthesized CT-LG is non-toxic to human cells, ingestible, and suitable for contact with food.

### Example 16. Biodegradation Properties of KBCS Composite Material in Soil

A soil burial degradation test was carried out to evaluate the biodegradability of the provided films. The biodegradation test was performed by burying a film pieces with dimensions of 5×5 cm in soil at a depth of 10 cm, as illustrated in FIG. 49. After 10, 20 and 30 days, film samples were collected, washed with distilled water, and placed in a 50° C. oven for 1 day. The weight loss of the film samples was monitored and recorded.

Over the first 10 days, both KBCS and KBCS-Alg-PEG200 films lost about 50% of their mass (FIG. 50). The films had become broken down into smaller pieces and become thinner. The observed transparency difference between the KBCS and KBCS-Alg-PEG200 films was no longer apparent, indicating that the alginate coating was completely biodegraded by soil microorganisms within 10 days. After 20 d, both the KBCS and KBCS-Alg-PEG200 films were further reduced to approximately 5-10% of their original weight, with some films disappearing and others degrading to multiple small pieces (FIG. 51). Finally, the films completely disappeared by 30 d, reflecting an efficient biodegradation process by the microorganisms in the soil.

In the first 10 days of the biodegradation test, BC/CT-LG films lost about 15% mass with the formation of nicks in the material (FIG. 52). After 20 d, both the films further decreased to 30% of their original weights (FIG. 52). The films completely disappeared after 2 months, reflecting efficient biodegradation by soil microorganisms. The provided composite materials therefore possess an eco-friendly life cycle, characterized by renewability and sustainability.

Although the foregoing disclosure has been described in some detail by way of illustration and example for purpose of clarity of understanding, one of skill in the art will appreciate that certain changes and modifications within the spirit and scope of the disclosure may be practiced, e.g., within the scope of the appended claims. It should also be understood that aspects of the disclosure and portions of various recited embodiments and features can be combined or interchanged either in whole or in part. In the foregoing descriptions of the various embodiments, those embodiments which refer to another embodiment may be appropriately combined with other embodiments as will be appreciated by one of skill in the art. Furthermore, those of ordinary skill in the art will appreciate that the foregoing description is by way of example only and is not intended to limit the disclosure. In addition, each reference provided herein is incorporated by reference in its entirety for all purposes to the same extent as if each reference was individually incorporated by reference.

1. A biodegradable film comprising:
a bacterial cellulose matrix;
a protein entrapped within the bacterial cellulose matrix; and
a crosslinked coating on the bacterial cellulose matrix, wherein the crosslinked coating comprises an ionic polysaccharide, a polyether, and a metal cation.

2. The biodegradable film of claim 1, wherein the protein comprises soy protein isolate.

3. The biodegradable film of claim 1, wherein the ionic polysaccharide comprises an alginate.

4. The biodegradable film of claim 1, wherein the polyether comprises polyethylene glycol.

5. The biodegradable film of claim 1, wherein the metal cation comprises calcium.

6. The biodegradable film of claim 1, wherein the concentration of the bacterial cellulose matrix within the biodegradable film is between about 76 wt % and about 85 wt %.

7. The biodegradable film of claim 1, wherein the concentration of the protein within the biodegradable film is between about 0.4 wt % and about 0.8 wt %.

8. The biodegradable film of claim 1, wherein the concentration of the ionic polysaccharide within the biodegradable film is between about 12 wt % and about 27 wt %.

9. The biodegradable film of claim 1, wherein the concentration of the polyether within the biodegradable film is between about 6 wt % and about 10 wt %.

10. The biodegradable film of claim 1, wherein the concentration of the metal cation within the biodegradable film is between about 1.0 wt % and about 2.4 wt %.

11. A biodegradable film comprising:
a bacterial cellulose matrix; and
a hydrophobic coating on the bacterial cellulose matrix, wherein the hydrophobic coating comprises a hydrophobically modified polysaccharide.

12. The biodegradable film of claim 11, wherein the hydrophobic coating comprises a hydrophobically modified chitosan.

13. The biodegradable film of claim 11, wherein the hydrophobic coating comprises a polysaccharide modified with a phenolic fatty acid ester.

14. The biodegradable film of claim 13, wherein the phenolic fatty acid ester comprises lauryl gallate.

15. The biodegradable film of claim 11, wherein the hydrophobic coating comprises a lauryl gallate modified chitosan.

16. The biodegradable film of claim 11, wherein the concentration of the hydrophobic coating within the biodegradable film is between about 10 wt % and about 45 wt %.

17. The biodegradable film of claim 11, wherein the molar ratio of the lauryl gallate to saccharide subunits of the chitosan is greater than about 0.7:1.

18. A packaging material comprising the biodegradable film of claim 1, wherein the packaging material is a food contact substance.

19. A drinking straw comprising the biodegradable film of claim 1.

20. A method of producing a biodegradable film, the method comprising:
culturing a symbiotic colony of bacteria and yeast (SCOBY) in a cultivation medium under culture conditions suitable for synthesis of a bacterial cellulose matrix entrapping protein, wherein the cultivation medium comprises a sugar, a tea, and a protein mixture including the protein;
harvesting the bacterial cellulose matrix;
coating the bacterial cellulose matrix with a coating solution under conditions suitable for forming coated bacterial cellulose, wherein the coating solution comprises an ionic polysaccharide and a polyether; and
contacting the coated bacterial cellulose with a crosslinking solution under conditions suitable for crosslinking the coated bacterial cellulose, wherein the crosslinking solution comprises a metal salt, thereby forming the biodegradable film.

21. A method of producing a biodegradable film, the method comprising:
culturing a symbiotic colony of bacteria and yeast (SCOBY) in a cultivation medium under culture conditions suitable for synthesis of a bacterial cellulose matrix, wherein the cultivation medium comprises a sugar and a tea;
harvesting the bacterial cellulose matrix; and
coating the bacterial cellulose matrix with a coating solution under conditions suitable for forming coated bacterial cellulose, wherein the coating solution comprises a hydrophobically modified polysaccharide.
